(19) United States
(12) Patent Application Publication
(10) Pub. No.: US 2014/0105855 A1
(43) Pub. Date: Apr. 17, 2014

(54) STRUCTURAL BASED DESIGN OF IL-17 DOMINANT NEGATIVE MUTANTS

(71) Applicant: Kenan Christopher Garcia, Menlo Park, CA (US)

(72) Inventor: Kenan Christopher Garcia, Menlo Park, CA (US)

(21) Appl. No.: 14/009,904

(22) PCT Filed: Apr. 6, 2012
(86) PCT No.: PCT/US12/32493

§ 371 Date: Dec. 16, 2013

Related U.S. Application Data
(60) Provisional application No. 61/472,372, filed on Apr. 6, 2011.

(51) Int. Cl. C07K 14/54

(52) U.S. Cl. CPC C07K 14/54
USPC 424/85.2; 530/351; 536/23.5; 435/252.3; 435/69.52; 435/252.33; 435/252.31; 435/252.34; 435/254.2; 435/254.21; 435/254.23; 435/325; 435/365.1; 435/357; 435/358; 435/367; 435/352; 435/364

(57) ABSTRACT

IL-17 Receptor binding proteins, including non-naturally occurring and recombinantly modified proteins, methods of making and using such molecules as therapeutic, prophylactic and diagnostic agents are provided.

This application claims priority to U.S. provisional patent application Ser. No. 61/472,372, filed Apr. 6, 2011, the contents of which are incorporated in their entirety.

### STATEMENT AS TO RIGHTS TO INVENTIONS MADE UNDER FEDERALLY SPONSORED RESEARCH OR DEVELOPMENT

This invention was made with U.S. Government support under Grant no. AI51321 awarded by the National Institutes of Health. The U.S. Government has certain rights in this invention.

### FIELD OF THE INVENTION

The present invention relates to IL-17 receptor binding proteins, and specifically to their uses in the prevention and/or treatment of acute and chronic immunological diseases such as rheumatoid arthritis, osteoarthritis, psoriasis, multiple sclerosis, and other autoimmune diseases.

### BACKGROUND

Cytokines are secreted soluble proteins with pleiotropic activities involved in immune and inflammatory responses. Cytokines bind to specific cell surface receptors, triggering signal transduction pathways that lead to cell activation, proliferation, and differentiation.

One such cytokine, interleukin-17 (IL-17), originally named “CTL-associated antigen 8” or “CTLA-8”, was isolated and cloned from murine hybridomas and shown to have homology to open reading frame 13 of the T lymphotropic Herpesvirus saimiri. Since then, five related cytokines that share 20-50% identity to IL-17 have been identified.

To indicate IL-17 as the founding member of the IL-17 cytokine family, it has been designated IL-17A; the other members have been designated IL-17B, IL-17C, IL-17D, IL-17E, and IL-17F. The human gene for IL-17A encodes a 155 amino acid polypeptide comprising a 19 amino acid signal sequence and a 132 amino acid mature domain.

IL-17 cytokine family members share conserved cysteine residues. Of interest are IL-17A and IL-17F, which share 50% identity; both cytokines are induced by IL-23, co-expressed by T cells, and considered potential targets for T cell-mediated autoimmune diseases. Similar to IL-17A, the conserved cysteine residues in IL-17F exhibit features of a classic cysteine knot motif found in bone morphogenetic proteins (BMPs), transforming growth factor-beta (TGF-β), nerve growth factor (NGF) and platelet-derived factor BB (PDGF-BB).

IL-17A can exist as either a homodimer or as a heterodimer complexed with IL-17F to form heterodimeric IL-17A/F. IL-17A and IL-17F share the same receptor (IL-17R), which is expressed on a wide variety of cells including vascular endothelial cells, peripheral T cells, B cells, fibroblast, lung cells, myelomonocytic cells, and marrow stromal cells.

IL-17A is involved in the induction of proinflammatory responses and induces or mediates expression of a variety of other cytokines, factors, and mediators including tissue necrosis factor-alpha (TNF-α), IL-6, IL-8, IL-1β, granulocyte colony-stimulating factor (G-CSF), prostaglandin E₂, IL-10, IL-12, IL-1R antagonist, leukemia inhibitory factor, and stromelysin. IL-17A also induces nitric oxide in chondrocytes and in human osteoarthritis explants.

Increased levels of IL-17A have been associated with several conditions, diseases or disorders including airway inflammation, rheumatoid arthritis (“RA”), osteoarthritis, bone erosion, intraperitoneal abscesses and adhesions, inflammatory bowel disorder (“IBD”), allograft rejection, psoriasis, certain types of cancer, angiogenesis, atherosclerosis and multiple sclerosis (“MS”).

Accordingly, a need exists for compounds that can effectively mediate or neutralize the activity of IL-17 in the inflammatory response and autoimmune disorders.

### SUMMARY OF THE INVENTION

Cytokines of the IL-17 family are central mediators of chronic inflammatory and autoimmune conditions. For example, increased levels of IL-17A have been associated with several conditions, diseases or disorders including airway inflammation, rheumatoid arthritis (“RA”), osteoarthritis, bone erosion, intraperitoneal abscesses and adhesions, inflammatory bowel disorder (“IBD”), allograft rejection, psoriasis, certain types of cancer, angiogenesis, atherosclerosis and multiple sclerosis (“MS”).

The IL-17 family is composed of six cytokines and five receptors. Within the IL-17 cytokine family, IL-17A and IL-17F share the greatest identity (50%) and are bound by IL-17 receptor A (IL-17RA, A001253) and IL-17RC. Both IL-17RA and IL-17RC are co-expressed by fibroblasts, epithelial and endothelial cells, while T cells only express IL-17RA.

Both IL-17A and IL-17F are produced by Th-17 cells.

Disclosed herein are compositions and methods for regulating the affects of IL-17 cytokines. In some embodiments, a nucleic acid molecule is disclosed that encodes an IL-17 heterodimer composed of three subunits. The first of these subunits is a wild-type IL-17 monomer, the second of these subunits is an IL-17 monomer mutein, and the third subunit is a linker that links the first and second subunit. Expression of this nucleic acid results in a heterodimeric IL-17 polypeptide (“IL-17w/m”).

In other embodiments, a method for preparing a heterodimeric IL-17 protein is disclosed. This method entails, obtaining an isolated host cell with a nucleic acid that encodes an IL-17w/m and growing the host cell under appropriate culture conditions and in appropriate medium so that the nucleic acid is expressed. The IL-17w/m protein is isolated from the host cell itself or the host cell culture medium.

In some embodiments, a pharmaceutical composition composed of an IL-17w/m is disclosed. In other embodiments, a method of administering an IL-17w/m protein is taught.

These and other embodiments, features and potential advantages will become apparent with reference to the following description and drawings.

### BRIEF DESCRIPTION OF THE DRAWINGS

FIG. 1 is a schematic illustration of the structure of the IL-17RA-IL-17F complex. Ribbon diagram of IL-17RA in bound to IL-17F (chain A and chain B), N-linked glycans are shown in ball-and-stick representation. IL-17RA is composed of two fibronectin type III domains (D1 and D2) joined by a short helical linker. The right-hand panel shows the complex rotated by 60° around the y-axis.

FIG. 2 is a schematic illustration demonstrating IL-17F binding to IL-17RA is mediated by three distinct interfaces. (A) Site 2, the IL-17RA D1 C-C′ loop inserts between the N-terminal coil region and strands 1 and 2 of the IL-17F chain B. The N-terminal coil undergoes a conformational change between the unbound and bound conformations. (B) Site 2, surface representation of the knob-in-holes IL-17F binding pocket complementarity. (C) Site 1, the IL-17RA D1 N-terminal binding site. (D) Site 3, the IL-17RA D2 binding site. Contact residues are shown as stick models. Dotted lines represent hydrogen bonds and salt-bridges.

FIG. 3 is an assembly and model of the heterodimeric IL-17 signaling complex. (A) IL-17 receptor-cytokine affinity was measured by surface plasmon resonance (SPR). IL-17RA, IL-17RB and IL-17RC were immobilized on the SPR chip surface, and the binding affinity of IL-17A, IL-17F or IL-17E was measured. Where indicated, the affinity of a second receptor binding to the pre-assembled receptor-cytokine complex on the chip was then measured. For kinetic experiments (top 3 rows), representative SPR sensorgrams are shown as colored lines and the curve-fit as a black line. Time in seconds (s) is plotted against response (RU, resonance units). The injected concentrations are to the right of the sensorgrams. For equilibrium experiments (fourth row), the injected concentration (M) is plotted against the maximum response (RU) for a representative experiment; the curve fit is shown as a black line and the dissociation constant (Kd) is marked as a vertical line. The insets show cartoon representations of the binding event. The Kd is reported as the mean of at least two independent experiments±the standard error of the mean. (B) Model of heterodimeric signaling complex formation. The second receptor was modeled assuming that both receptors bind to IL-17F in the same orientation. The C-terminal domains (D2) of the receptors come into close proximity as highlighted by the box.

FIG. 4 is a schematic illustration demonstrating the binding interface and conserved IL-17 residues. Surface representation of IL-17F with IL-17RA in ribbon format. (A) IL-17RA-IL-17F contact residues. (B) Residues conserved among IL-17A and IL-17F are mapped onto the IL-17F structure; identical residues are stippled. (C) Residues identical among 4, 5 or 6 IL-17 cytokine family members are indicated and conservative substitutions across all six cytokines are also identified. (D) Alignment of human IL-17 cytokines. Residues that form contacts in the IL-17RA-IL-17F structure are highlighted by a black box on the IL-17F sequence and underneath the alignment. Residues that are identical in four, five or six cytokines are stippled; those identical in all six cytokines are also marked with ‘*’; conserved groups are marked with ‘:’. The sequences correspond to SEQ ID NOs:12, 2, 6, 8, 10, and 4, respectively.

FIG. 5 is a comparison of the IL-17RA-IL-17F receptor complex compared to homodimeric cysteine-knot growth factor receptor complexes. (A) IL-17RA-IL-17F, (B) P75NTR-NGF and (C) TrkA-NGF are shown as ribbon models.

### DETAILED DESCRIPTION

In order for the present invention to be more readily understood, certain terms and phrases are defined below as well as throughout the specification.

### DEFINITIONS

The term “effective amount” as used herein refers to the amount necessary to elicit a desired biological response. The effective amount of a drug may vary depending on such factors as the desired biological endpoint, the drug to be delivered, the composition of any additional active or inactive ingredients, etc.

The term “expression” is used herein to mean the process by which a polypeptide is produced from a nucleic acid. When the nucleic acid is DNA, this process involves the transcription of the gene into mRNA and the translation of this mRNA into a polypeptide. Depending on the context in which it is used, “expression” may refer to the production of RNA, protein, or both.

The term “gene product” as used herein means an RNA (for example, a messenger RNA (mRNA) or a micro RNA (miRNA)) or protein that is encoded by the gene.

As used herein, the term “isolated” refers to a molecule that is substantially pure. An isolated protein may be substantially pure if it is, for example, 55%, 60%, 65%, 70%, 75%, 80%, 85%, 95%, 98%, 99% or more free of a polypeptide or polypeptides with less than 55%, 60%, 65%, 70%, 75%, 80%, 85%, 95%, 98%, 99% identity to the protein to be isolated.

As used herein, the terms “modulate” or “modulation” can refer to the downregulation (i.e., inhibition, suppression, decrease, or reduction), of specifically targeted genes (including their RNA and/or protein products), signaling pathways, cells, and/or a targeted phenotype. For example, “modulate” and “modulation” can refer to downregulation of IL-17 receptor signaling. “Modulate” or “modulation” can also refer to the upregulation (i.e., induction or increase) of the targeted genes.

Downregulation refers to a suppression of 2%, 5%, 10%, 15%, 20%, 25%, 30%, 35%, 40%, 45%, 50%, 55%, 60%, 65%, 70%, 75%, 80%, 85%, 90%, 95% or more between an experimental condition and a control condition, for example.

Upregulation refers to a increase of 2%, 5%, 10%, 15%, 20%, 25%, 30%, 35%, 40%, 45%, 50%, 55%, 60%, 65%, 70%, 75%, 80%, 85%, 90%, 95% or 2-fold, 3-fold, 5-fold, 10-fold, 20-fold, 50-fold, 100-fold, 1000-fold or more between an experimental condition and a control condition.

“Patient” or “subject” means a mammal, e.g. a human, who has or is at risk for developing a disease or condition such as an inflammatory disease, or has or is diagnosed as having an inflammatory disease, or could otherwise benefit from the compositions and methods described herein.

The terms “treating” or “treatment” or “alleviation” or “amelioration” refer to both therapeutic treatment and prophylactic or preventative measures, wherein the object is to prevent or slow down (lessen) the targeted pathologic condition or disorder.

The term “IL-17 receptor” refers to proteins that bind to an IL-17 cytokine such as IL-17RA, IL-17RB, IL-17RC, IL-17RD, and IL-17RE receptors, particularly human isoforms of these receptors and extracellular domains of these receptors.

### Immomodulatory Polypeptides

Naïve T cells are stimulated to differentiate into specialized effector cells primarily through the actions of secreted cytokines. T helper (Th) cells have been typically considered to fall into one of two effector cell lineages; Th1 and Th2 cells modulating cellular and humoral T cell immunity, respectively, based on their cytokine expression profiles. More recent work described Th17 cells, a third lineage of effector Th cells distinct from, and in fact antagonized by products of the Th1 and Th2 lineages. Named after their signature cytokine interleukin 17 (IL-17), this subset of Th cells appear to have evolved as an arm of the adaptive immune system specialized for enhanced host protection against extracellular bacteria and some fungi, as these microbes may not be effectively controlled by Th1 or Th2 responses.

The varied tissue sources of cytokines that induce differentiation and regulate homeostasis of Th17 cells, namely IL-23, IL-6, and transforming growth factor-13 (TGF-β), together with the presence of IL-17 receptors on both hematopoietic and non-hematopoietic cells, have highlighted the complicated relationships that exist between adaptive and innate immune cells. While the full scope of Th17 cell effector functions is still emerging, the strong inflammatory response promoted by Th17 cells has been associated with the pathogenesis of a number of autoimmune and inflammatory disorders previously attributed to Th1 or Th2 cells including rheumatoid arthritis, multiple sclerosis and psoriasis.

In addition to IL-17A, members of the IL-17 family include IL-17B, IL-17C, IL-17D, IL-17E (also termed IL-25), and IL-17F. All members of the IL-17 family have a similar protein structure including four highly conserved cysteine residues. IL-17A and F are most closely related followed by IL-17B (29%), IL-17D (25%), IL-17C (23%), and IL-17E being most distantly related to IL-17A (17%).

These cytokines are all well conserved in mammals, with as much as 62-88% of amino acids conserved between the human and mouse homologs. There is no sequence similarity to other cytokines.

On the basis of the crystal structure of IL-17F, the six structurally related IL-17 cytokines (IL-17A-IL-17F) are predicted to form a homodimeric fold (or heterodimeric fold in the case of IL-17A-F) homologous to that of the cysteine-knot growth factors such as nerve growth factor (NGF).

Th17 cell-derived IL-17A and IL-17F share the greatest homology within the family and require both IL-17RA and TL-17RC for signaling. While it has been shown that fibroblasts, epithelial and endothelial cells coexpress both IL-17RA and IL-17RC, T cells do not demonstrably express IL-17RC, and only express IL-17RA. Thus,

The IL-17 family of cytokines, in part through their actions as effector cytokines of the Th17 lineage, provides innovative approaches to the manipulation of immune and inflammatory responses. As such, antagonists of IL-17A, IL-17B, IL-17C, IL-17D, IL-17E, IL-17F, and their receptors, either singly or together, such as antagonists described herein, are useful in therapeutic treatment of inflammatory diseases such as multiple sclerosis, inflammatory bowel disease (IBD), rheumatoid arthritis, psoriasis, and cancer. Moreover, antagonists of IL-17 family member activity, such as antagonists described herein, are useful in therapeutic treatment of other inflammatory diseases.

The five IL-17 receptors (IL-17RA-IL-17RE) are not homologous to any known receptors, and exhibit considerable sequence divergence. Signaling competent IL-17 receptors are likely homo- or hetero-dimers. All appear to contain extracellular domains composed of fibronectin type-III (FnIII) domains, and cytoplasmic SEF/IL-17R (SEFIR) domains that show loose homology to Toll/IL-1R (TLR) domains.

The IL-17 receptors mediate signaling events that are distinct from those triggered by the more widely known receptors for type I four helix cytokines. Like TLR stimulation, IL-17 receptor stimulation results in activation of NF-κB and mitogen-activated protein kinases (MAPK). However, IL-17 receptor signaling does not utilize the same set of membrane proximal adaptor molecules as TLR signaling; IL-17R requires the adaptor Act1 which also contains a SEFIR domain. These unique signaling properties of IL-17 receptors enable Th-17 cells to act as a bridge between innate and adaptive immune cells.

Thus, for epithelial cells, for example, the binding of homo- or hetero-dimeric IL-17A and/or IL-17F to the hetero-dimeric receptor IL-17RA/RC leads to the recruitment of Act1. This is turn allows the incorporation of TRAF6 into the signaling complex and then ‘downstream’ activation of the NF-κB and mitogen-activated protein kinase pathways.

In some embodiments, provided are binding peptides, proteins, and any fragments or permutations thereof that bind to an IL-17R or an IL-17 cytokine referred to interchangeably as “IL-17R antagonists”, “IL-17 antagonists”, “IL-17R neutralizing entities”, “IL-17R designer cytokine antagonists”, and “IL-17 designer cytokine antagonists.”

Specifically, in some embodiments, such binding peptides or proteins are capable of specifically binding to a human IL-17R and are referred to as “IL-17R binding proteins.” Further, these binding peptides or proteins are capable of modulating biological activities associated with IL-17, e.g., antagonizing IL-17 activation of an IL-17 receptor, and thus are useful in the treatment of various diseases and pathological conditions such as inflammation and immune-related diseases. Exemplary antagonists have an IC50 of less than 200, 50, 20, or 10 nM.

In still other embodiments, the invention concerns an isolated polynucleotide that encodes a polypeptide of the present invention, wherein said polypeptide is capable of binding to IL-17R, e.g., IL-17RA, IL-17RB, IL-17RC, IL-17RD, or IL-17RE, and reducing its signaling capability.

The present invention also provides protein conjugates comprising an antagonist of the present invention conjugated to a polymer of polyethylene glycol.

The present invention further includes pharmaceutical compositions, comprising a pharmaceutically acceptable carrier and an IL-17R antagonist described herein.

In another aspect, the invention concerns a method for the treatment of an inflammatory disease characterized by elevated expression of IL-17 and/or IL-23 and/or IFN-γ in a mammalian subject, comprising administering to the subject an effective amount of an antagonist of IL-17 signaling.

Typical methods of the invention include methods to treat pathological conditions or diseases in mammals associated with or resulting from increased or enhanced IL-17 and/or IL-23 and/or IFN-γ expression and/or activity. In the methods of treatment, the antagonists of the present invention may be administered which preferably reduce the respective receptor activation. The methods contemplate the use of an antagonist of IL-17R that reduces signaling by blocking IL-17R complex formation.

Antagonists of the present invention (e.g., antagonists of IL-17R) are also useful to prepare medicines and medicaments for the treatment of immune-related and inflammatory diseases, including for example, systemic lupus erythematosis, arthritis, rheumatoid arthritis, osteoarthritis, psoriasis, demyelinating diseases of the central and peripheral nervous systems such as multiple sclerosis, idiopathic demyelinating polyneuropathy or Guillain-Barre syndrome, inflammatory bowel disease, colitis, ulcerative colitis, Crohn's disease, gluten-sensitive enteropathy, autoimmune ocular diseases, cancer, neoplastic diseases, atherosclerosis, and angiogenesis.

In a specific aspect, such medicines and medicaments comprise a therapeutically effective amount of an IL-17R antagonist with a pharmaceutically acceptable carrier. Preferably, the admixture is sterile.

Processes for producing the same are also herein described, wherein those processes comprise culturing a host cell comprising a vector which contains the appropriate encoding nucleic acid molecule under conditions suitable for expression of said antibody and recovering said antibody from the cell culture.

### IL-17R Binding Proteins

An IL-17 cytokine can include at least three sites that contact an IL-17R on one of its receptor binding faces. The IL-17 generally includes two subunits (here designated Chain A and B), each contributing amino acids to a particular receptor binding face. The use of the terms “Chain A” and “Chain B” is merely for reference. For example, in embodiments using a single chain format, “Chain A” may be placed C-terminal to “Chain B” and alternatively it may be place N-terminal to “Chain B.”

The IL-17 interface that binds IL-17RA includes three sites (Site 1, Site 2, and Site3).

In one aspect, this disclosure features an IL-17R binding protein that comprises an IL-17 cytokine including two subunits wherein one receptor binding face of the dimer formed by the two subunits includes one or more substitutions, e.g., at least two or three substitutions, e.g., non-conservative substitutions or a substitutions described herein.

TABLE 1

| Chain “A” Position | |
|---|---|
| (numbering based on | |
| the IL-17F sequence) | Chain “B” Position |
| N89 | |
| Q95 | R41 |
| | R42 |
| | R47 |
| | Y63 |
| | V68 |

For example, the cytokine has at least one, two, three, four, five, six, or seven substitutions (or deletions) at the positions identified in Table 1 above, e.g. between two to seven, or three to seven, or three to six. In some cases, one cytokine subunit differs from the other subunit by at one, two, three, four, five, six, or seven substitutions (or deletions). For example, in the IL-17R binding protein, the two receptor binding faces can include different amino acids, e.g., at least one, two, three, four, five, six, or seven differences, e.g., at positions corresponding to those in Table 1.

One or both the subunits can have one or more conservative and/or one or more non-conservative substitutions. Typically, at least one subunit or both subunits are at least 90, 92, 94, 95, 96, 97, or 98% identical, but not 100% identical to a mature human IL-17, e.g., SEQ ID NO:2, 4, 6, 8, 10, 12, or 20. In one embodiment, neither subunit is 100% identical to a mature human IL-17, e.g., they differ by at least one, two, or three amino acids from the human IL-17 from which they were derived. In one embodiment, one subunit differs from a mature human IL-17, whereas the other subunit is identical to a mature human IL-17. In certain embodiments, the substitutions in a subunit are not to residues in a corresponding murine protein.

In one embodiment, an IL-17R binding protein comprises an IL-17 cytokine including two subunits in which Site 2 of one receptor binding face includes one or more mutations, e.g., at least two or three mutations, e.g., non-conservative mutations

In one embodiment, an IL-17R binding protein comprises an IL-17 cytokine including two subunits in which Site 3 of one receptor binding face includes one or more mutations, e.g., at least two or three mutations, e.g., non-conservative mutations

Exemplary IL-17R binding proteins include a plurality of mutations, for example: at least one, two, or three substitutions in Site 1 and at least one, two or three substitutions in Site 2; at least one, two, or three substitutions in Site 1 and at least one, two or three substitutions or deletions in Site 3; at least one, two, or three substitutions in Site 2 and at least one, two or three substitutions or deletions in Site 3; at least one, two, or three substitutions in Site 1, at least one, two, or three mutations in Site 2, and at least one, two or three substitutions or deletions in Site 3.

Exemplary IL-17R binding proteins include a plurality of substitutions and/or deletions in an IL-17 cytokine. Sequences that are at least 85, 90, 92, 94, 96, 98, or 99% identical to such sequences and that include substitutions at the same positions as such sequences may also be used.

In one embodiment, an IL-17R binding protein is used as a receptor antagonist, e.g., to bind to an IL-17 receptor subunit and prevent receptor dimerization.

### Amino Acid Modifications

Polypeptides described herein can be modified in a variety of ways including substitution, deletion, or addition. A substitution entails the replacement of one amino acid for another. Such replacements can be made using any one of the twenty amino acids directly encoded by the genetic code: alanine, arginine, asparagine, aspartic acid, cysteine, glutamic acid, glutamine, glycine, histidine, isoleucine, leucine, lysine, methionine, phenylalanine, proline, serine, threonine, tryptophan tyrosine, and valine. In addition, amino acids of a polypeptide can be replaced using amino acids not directly encoded by the genetic code for example: selenocysteine, pyrrolysine, p-nitrophenylalanine, p-sulfotyrosine, p-carboxyphenylalanine, o-nitrophenylalanine, 5-nitro His, 3-nitro Tyr, 2-nitro Tyr, nitro substituted Leu, nitro substituted His, nitro substituted Ile, nitro substituted Trp, 2-nitro Trp, 4-nitro Trp, 5-nitro Trp, 6-nitro Trp, 7-nitro Trp, aminotyrosines, and carboxyphenyalanines.

Conservative amino acid substitutions can frequently be made in a protein without altering either the conformation or the function of the protein. Substitutions can be chosen based on their potential effect on (a) backbone structure in the vicinity of the substitution, for example, a sheet or helical conformation, (b) the charge or hydrophobicity of the molecule at the target site, or (c) the volume and branching of the side chain.

Amino acid residues can be classified based on side-chain properties: (1) aliphatic: ala, met, val, leu, ile; (2) small aliphatic: ala, val; (3) large aliphatic: met, leu, ile; (4) neutral hydrophilic: ser, thr; asn; gln; (5) acidic: asp, glu; (6) basic: his, lys, arg; (7) charged: arg, asp, glu, his, lys; (8) residues that affect backbone conformation: gly, pro; and (9) aromatic: trp, tyr, phe. Non-conservative substitutions can include substituting a member of one of these classes for a member of a different class or making a substitution not identified in the table below. Conservative substitutions can include substituting a member of one of these classes for another member of the same class.

### Heterodimer Formation

Any appropriate approach can be used to form heterodimers of two cytokine subunits described herein. Exemplary heterodimers include heterodimers of two different sequence variants of IL-17A, IL-17F, IL-17B, IL-17C, IL-17D, and IL-17E, as well as heterodimers that combine two different cytokine family members, e.g., a sequence variant of IL-17A and a wildtype or variant of IL-17F; a sequence variant of IL-17F and a wildtype or variant of IL-17A; and so forth.

An approach to forming heterodimers is to connect the two subunits using a linker to form a single chain protein. The linker can be any appropriate length, e.g., 6, 7, 8, 9, 10, 11, 12, 13, 14, 15, 16, 17, 18, 19, 20, 21, 22, 23, 24, 25, 27, 29, 30, 31, 32, 33, 34, 35, or more residues, e.g., between 25-34 or 27-37 residues. The linker can include a repeating sequence having the formula [(gly)_{B}(ser)]_{X}, wherein B is an integer from 1-5 and X is an integer from 1-20. Longer and shorter linkers can also be used. Linker lengths with maximum stability and maximum heterodimer formation can be selected and used.

IL-17R binding proteins and other proteins described herein can be produced by expression in recombinant host cells, but also by other methods such as in vitro transcription and translation and chemical synthesis. For cellular expression, one or more nucleic acids (e.g., cDNA or genomic DNA) encoding a binding protein may be inserted into a replicable vector for cloning or for expression. Various vectors are publicly available.

The vector may, for example, be a plasmid, cosmid, viral genome, phagemid, phage genome, or other autonomously replicating sequence. The appropriate coding nucleic acid sequence may be inserted into the vector by a variety of procedures. For example, appropriate restriction endonuclease sites can be engineered (e.g., using PCR). Then restriction digestion and ligation can be used to insert the coding nucleic acid sequence at an appropriate location. Vector components generally include one or more of an origin of replication, one or more marker genes, an enhancer element, a promoter, and a transcription termination sequence.

For bacterial expression, the binding protein can be produced with or without a signal sequence. For example, it can be produced within cells so that it accumulates in inclusion bodies. It can also be secreted, e.g., by addition of a prokaryotic signal sequence, e.g., an appropriate leader sequence such as from alkaline phosphatase, penicillinase, or heat-stable enterotoxin II. Exemplary bacterial host cells for expression include any transformable E. coli K-12 strain (such as E. coli C600, ATCC 23724; E. coli HB101 NRRLB-11371, ATCC-33694; E. coli MM294 ATCC-33625; E. coli W3110 ATCC-27325), strains of B. subtilis, Pseudomonas, and other bacilli. Proteins produced in bacterial systems will typically lack glycosylation.

The binding protein can be expressed in a yeast host cell, e.g., Saccharomyces cerevisiae, Schizosaccharomyces pombe, Hanseula, or Pichia pastoris. For yeast expression, the binding protein can also be produced intracellularly or by secretion, e.g., using the yeast invertase leader or alpha factor leader.

In mammalian cell expression, mammalian signal sequences may be used to direct secretion of the protein, such as signal sequences from secreted polypeptides of the same or related species, as well as viral secretory leaders. Expression vectors used in eukaryotic host cells (yeast, fungi, insect, plant, animal, human, or nucleated cells from other multicellular organisms) can also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding the binding protein. The expression vector may also include one or more intronic sequences.

The binding protein can also be expressed in insect cells, e.g., Sf9 or SF21 cells, e.g., using the pFAST-BAC™ system.

The binding protein can also be expressed in mammalian cells. For example, cell lines of mammalian origin also may be employed. Examples of mammalian host cell lines include the COS-7 line of monkey kidney cells (ATCC CRL 1651) (Gluzman et al., Cell 23:175, 1981), L cells, C127 cells, 3T3 cells (ATCC CCL 163), Chinese hamster ovary (CHO) cells, HeLa cells, and BHK (ATCC CRL 10) cell lines, and the CV1/EBNA cell line derived from the African green monkey kidney cell line CV1 (ATCC CCL 70) as described by McMahan et al. (EMBO J. 10: 2821, 1991). Established methods for introducing DNA into mammalian cells have been described (Kaufman, R. J., Large Scale Mammalian Cell Culture, 1990, pp. 1569).

Still other methods, vectors, and host cells suitable for adaptation to the synthesis of binding protein in recombinant cells are described in Molecular Cloning: A Laboratory Manual, Third Ed., Sambrook et al. (eds.), Cold Spring Harbor Press, (2001) (ISBN: 0879695773). IL-17 cytokine proteins can be expressed and purified by any appropriate method, e.g., in mammalian, fungal, or bacterial cells. The proteins can be glycosylated or not glycosylated.

Once expressed in cells, IL-17R binding proteins and proteins described herein can be recovered from culture medium, inclusion bodies, or cell lysates. Cells can be disrupted by various physical or chemical means, such as freeze-thaw cycling, sonication, mechanical disruption, or cell lysing agents (e.g., detergents). IL-17R binding proteins and proteins described herein can be purified from other cell proteins or polypeptides that can be found in cell lysates or in the cell medium.

One exemplary purification procedure includes cation exchange chromatography and gel filtration e.g., Murphy et al. Protein Expr Purif 1998 March; 12(2):208-14. Various methods of protein purification may be employed and such methods are known in the art and described for example in Deutscher, Methods in Enzymology, 182 (1990); and Scopes, Protein Purification: Principles and Practice, Springer-Verlag, New York (2010) (ISBN: 1441928332). Purification moieties (such as epitope tags and affinity handles) can be optionally removed by proteolytic cleavage.

### Methods of Use

The compositions described herein are useful in methods for treating or preventing a disease or disorder in a vertebrate subject. In one such method, the step of administering to the subject a composition containing one or more polypeptides is provided. As described herein, the composition is administered intravascularly, topically, orally, rectally, ocularly, optically, nasally, or via inhalation.

A level of an inflammatory cytokine can be reduced upon the administration of a modified polypeptide in a mammalian subject, such as by administering to the subject a therapeutically effective amount of a composition comprising a modified IL-17. Exemplary inflammatory cytokines are IL-1, IL-6, TNF-α, IL-17, IL-21, and IL-23.

The level of inflammatory cytokine present in the blood and/or another tissue of the mammal is generally reduced. Modulation of the immune system also includes methods of increasing a level of an anti-inflammatory cytokine in a mammalian subject. For example, the anti-inflammatory cytokine is IL-10, IL-4, IL-11, IL-13, or TGF-β. Optionally, the level of the anti-inflammatory cytokine present in the blood of the mammal is increased.

In some aspects, an IL-17R binding protein or other engineered protein described herein is administered to a subject to treat a Th17 mediated disorder or a disorder mediated by an IL-17 cytokine family member. For example, the protein can be administered to a subject to treat atopic and contact dermatitis, colitis, endotoxemia, arthritis, rheumatoid arthritis, psoriatic arthritis, autoimmune ocular diseases (uveitis, scleritis), adult respiratory disease (ARD), demyelinating diseases, septic shock, multiple organ failure, inflammatory lung injury such as asthma, chronic obstructive pulmonary disease (COPD), airway hyper-responsiveness, chronic bronchitis, allergic asthma, psoriasis, eczema, IBS and inflammatory bowel disease (IBD) such as ulcerative colitis and Crohn's disease, diabetes, Helicobacter pylori infection, intra-abdominal adhesions and/or abscesses as results of peritoneal inflammation (i.e. from infection, injury, etc.), systemic lupus erythematosus (SLE), multiple sclerosis, systemic sclerosis, nephrotic syndrome, organ allograft rejection, graft vs. host disease (GVHD), kidney, lung, heart, etc. transplant rejection, streptococcal cell wall (SCW)-induced arthritis, osteoarthritis, gingivitis/periodontitis, herpetic stromal keratitis, restenosis, Kawasaki disease, and cancers/neoplastic diseases that are characterized by IL-17 and/or IL-23 expression, including but not limited to prostate, renal, colon, ovarian and cervical cancer, and leukemias (Tartour et al, Cancer Res. 5P:3698 (1999); Kato et al, Biochem. Biophys. Res. Commun. 282:735 (2001); Steiner et al, Prostate. 56:171 (2003); Langowksi et al, Nature 442: 461, 2006). For example, the binding protein is capable of binding, blocking, inhibiting, reducing, antagonizing or neutralizing IL-17 family members (either individually or together).

The compositions described herein may be used therapeutically or prophylactically. Cocktails of various different polypeptides can be used together to bind to and act upon one or multiple targets, e.g., multiple cell types, at once. Successful treatment can be assessed by routine procedures familiar to a physician.

In one embodiment, an IL-17R binding protein or described herein is administered to treat ocular disorders, including ocular disorders affecting the surface of the eye, ocular disorders mediated at least in part by an autoimmune reaction, ocular disorders associated with a systemic autoimmune disorder (such as Sjögren's syndrome and rheumatoid arthritis) or with a disorder associated with an IL-17 cytokine family member. The patient may or may not have other manifestations of a more systemic autoimmune disorder.

### Formulations

One or more therapeutic agent, alone or in combination with one or more chemotherapeutic agents, can be formulated with a pharmaceutically acceptable carrier for administration to a subject. The active ingredients can be formulated alone (individually) for sequential administration or may be formulated together for concurrent administration.

The term “pharmaceutically acceptable carrier” as used herein means one or more compatible solid or liquid filler, diluents or encapsulating substances which are suitable for administration to a subject. The components of the pharmaceutical compositions also are capable of being commingled with each other, in a manner such that there is no interaction, which would substantially impair the desired pharmaceutical efficiency. Such preparations may routinely contain pharmaceutically acceptable concentrations of salt, buffering agents, preservatives, compatible carriers, adjuvants and optionally other therapeutic ingredients.

The compositions described herein may be administered as a free base or as a pharmaceutically acceptable salt. Such pharmacologically and pharmaceutically acceptable salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene sulphonic, and benzene sulphonic. Also, pharmaceutically acceptable salts can be prepared as alkaline metal or alkaline earth salts, such as sodium, potassium or calcium salts of the carboxylic acid group.

The pharmaceutical compositions also may comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Suitable liquid or solid pharmaceutical preparation forms are, for example, aqueous or saline solutions for inhalation, microencapsulated, encochleated, coated onto microscopic gold particles, contained in liposomes (including pH-dependent release formulations), lipidoids, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of the compositions, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990 and Langer and Tirrell, Nature, 2004 Apr. 1; 428(6982): 487-92.

The compositions may conveniently be presented in unit dosage form and may be prepared by any of the methods well known in the art of pharmacy. In certain embodiments, the composition that is administered is in powder or particulate form rather than as a solution. Examples of particulate forms contemplated as part of the invention are provided in U.S. 2002/0128225. In some embodiments, the compositions are administered in aerosol form. In other embodiments, the compositions may be in powder form for constitution with a suitable vehicle, e.g., sterile pyrogen-free water, before use.

In addition, the compositions described herein may be formulated as a depot preparation, time-release, delayed release or sustained release delivery system. Such systems can avoid repeated administrations of the compositions described herein, increasing convenience to the subject and the physician. Such long acting formulations may be formulated with suitable polymeric or hydrophobic materials (for example as an emulsion in an acceptable oil) or ion exchange resins, or as sparingly soluble derivatives, for example, as a sparingly soluble salt.

Many types of release delivery systems are available and known to those of ordinary skill in the art. They include polymer based systems such as polylactic and polyglycolic acid, beta-glucan particles, polyanhydrides and polycaprolactone; nonpolymer systems that are lipids including sterols such as cholesterol, cholesterol esters and fatty acids, neutral fats such as mono-, di- and triglycerides or lipidoids; hydrogel release systems; silastic systems; peptide based systems; wax coatings, compressed tablets using conventional binders and excipients, partially fused implants and the like. In addition, a pump-based hardware delivery system can be used, some of which are adapted for implantation.

Controlled release can also be achieved with appropriate excipient materials that are biocompatible and biodegradable. These polymeric materials which effect slow release may be any suitable polymeric material for generating particles, including, but not limited to, non-biodegradable/non-biodegradable and biodegradable/biodegradable polymers. Such polymers have been described in great detail in the prior art and include, but are not limited to: beta-glucan particles, polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulfate sodium salt, poly(methyl methacrylate), poly(ethylmethacrylate), poly(butylmethacrylate), poly(isobutylmethacrylate), poly(hexylmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), poly(vinyl acetate, poly vinyl chloride polystyrene, polyvinylpryrrolidone, hyaluronic acid, and chondroitin sulfate. In one embodiment the slow release polymer is a block copolymer, such as poly(ethylene glycol) (PEG)/poly(lactic-co-glycolic acid) (PLGA) block copolymer.

Examples of non-biodegradable polymers include ethylene vinyl acetate, poly(meth)acrylic acid, polyamides, copolymers and mixtures thereof.

Examples of biodegradable polymers include synthetic polymers, for example, beta-glucan particles, polymers of lactic acid and glycolic acid, polyanhydrides, poly(ortho)esters, polyurethanes, poly(butic acid), poly(valeric acid), poly(caprolactone), poly(hydroxybutyrate), poly(lactide-co-glycolide) and poly(lactide-co-caprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion. The foregoing materials may be used alone, as physical mixtures (blends), or as co-polymers. Preferred polymers are polyesters, polyanhydrides, polystyrenes and blends thereof.

Effective amounts of the compositions described herein are administered to a subject in need of such treatment. Effective amounts are those amounts, which will result in a desired improvement in the condition, disease or disorder or symptoms of the condition, disease or disorder.

Effective doses range from 1 ng/kg to 100 mg/kg body weight, or from 100 ng/kg to 50 mg/kg body weight, or from 1 μg/kg to 10 mg/kg body weight, depending upon the mode of administration. Alternatively, effective doses can range from 3 micrograms to 14 milligrams per 4 square centimeter area of cells. The absolute amount will depend upon a variety of factors (including whether the administration is in conjunction with other methods of treatment, the number of doses and individual patient parameters including age, physical condition, size and weight) and can be determined with routine experimentation. One useful dose that can be is the highest safe dose according to sound medical judgment.

The time between the delivery of the various active agents can be defined rationally by first principles of the kinetics, delivery, release, agent pharmacodynamics, agent pharmacokinetics, or any combination thereof. Alternatively, the time between the delivery of the various agents can be defined empirically by experiments to define when a maximal effect can be achieved.

### Mode of Administration

The mode of administration may be any medically acceptable mode including oral administration, sublingual administration, intranasal administration, intratracheal administration, inhalation, ocular administration, topical administration, transdermal administration, intradermal administration, rectal administration, vaginal administration, subcutaneous administration, intravenous administration, intramuscular administration, intraperitoneal administration, intrasternal, administration, or via transmucosal administration. In addition, modes of administration may be via an extracorporeal device and/or tissue-penetrating electro-magnetic device.

The particular mode selected will depend upon the particular active agents selected, the desired results, the particular condition being treated and the dosage required for therapeutic efficacy. The methods described herein, generally speaking, may be practiced using any mode of administration that is medically acceptable, for example, any mode that produces effective levels of inflammatory response alteration without causing clinically unacceptable adverse effects.

The compositions can be provided in different vessels, vehicles or formulations depending upon the disorder and mode of administration. For example, for oral application, the compositions can be administered as sublingual tablets, gums, mouth washes, toothpaste, candy, gels, films, etc.; for ocular application, as eye drops in eye droppers, eye ointments, eye gels, eye packs, as a coating on a contact lens or an intraocular lens, in contacts lens storage or cleansing solutions, etc.; for topical application, as lotions, ointments, gels, creams, sprays, tissues, swabs, wipes, etc.; for vaginal or rectal application, as an ointment, a tampon, a suppository, a mucoadhesive formulation, etc.

The compositions, may be administered by injection, e.g., by bolus injection or continuous infusion, via intravenous, subcutaneous, intramuscular, intraperitoneal, intrasternal routes. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. For oral administration, the compositions can be formulated readily by combining the compositions with pharmaceutically acceptable carriers well known in the art, e.g., as a sublingual tablet, a liquid formulation, or an oral gel.

For administration by inhalation, the compositions may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compositions and a suitable powder base such as lactose or starch. Medical devices for the inhalation of therapeutics are known in the art. In some embodiments the medical device is an inhaler. In other embodiments the medical device is a metered dose inhaler, diskhaler, Turbuhaler, diskus or a spacer. In certain of these embodiments the inhaler is a Spinhaler (Rhone-Poulenc Rorer, West Malling, Kent). Other medical devices are known in the art and include Inhale/Pfizer, Mannkind/Glaxo and Advanced Inhalation Research/Alkermes.

The compositions may also be formulated in rectal or vaginal compositions such as suppositories or retention enemas, e.g., containing conventional suppository bases such as cocoa butter or other glycerides.

### Additional Uses

A binding protein described herein can be labeled directly or indirectly with a moiety that is a label or produces a signal, e.g., an enzyme, a radiolabel, an epitope, or a fluorescent protein (such as green fluorescent protein). The binding protein can be contacted to a sample or to cells to determine if a receptor is present in the sample or on the cells, e.g., using standard immunoblotting, immunofluorescence, enzyme immunoassay (EIA), radioimmunoassay (RIA), fluorescence energy transfer, Western blot, and other diagnostic and detection techniques.

The binding protein can also be labeled for in vivo detection and administered to a subject. The subject can be imaged, e.g., by NMR or other tomographic means. For example, the binding agent can be labeled with a radiolabel such as 131I, 111In, 123I, 99mTc, 32P, 125I, 3H, 14C, and 188Rh, fluorescent labels such as fluorescein and rhodamine, nuclear magnetic resonance active labels, positron emitting isotopes detectable by a positron emission tomography (“PET”) scanner, chemiluminescers such as luciferin, and enzymatic markers such as peroxidase or phosphatase. The binding protein can be labeled with a contrast agent such as paramagnetic agents and ferromagnetic or superparamagnetic (which primarily alter T2 response)

A binding protein can also be used to purify cells which express the receptor to which it binds. For example, the binding protein can be coupled to an immobilized support (e.g., magnetic beads or a column matrix) and contacted to cells which may express the receptor. The support can be washed, e.g., with a physiological buffer, and the cells can be recovered from the support.

A binding protein can also be used to purify soluble forms of the receptor to which it binds. For example, samples containing the soluble receptor can be contacted to immobilized binding protein and then, e.g., after washing, can be recovered from the immobilized binding protein.

For example, the binding protein can be coupled to a radioactive isotope such as an α, β, or γ-emitter. Examples of radioactive isotopes include iodine (¹³¹I or ¹²⁵I), yttrium (⁹⁰Y), lutetium (¹⁷⁷Lu), actinium (²²⁵Ac), (praseodymium, or bismuth (²¹²Bi or ²¹³Bi). The binding protein can be coupled to a biological protein, a molecule of plant or bacterial origin (or derivative thereof), e.g., a maytansinoid (e.g., maytansinol, an analog thereof or DM1), as well as a taxane (e.g., taxol or taxotere), or a calicheamicin. Examples of maytansinol analogues include those having a modified aromatic ring (e.g., C-19-decloro, C-20-demethoxy, C-20-acyloxy) and those having modifications at other positions (e.g., C-9-CH, C-14-alkoxymethyl, C-14-hydroxymethyl or aceloxymethyl, C-15-hydroxy/acyloxy, C-15-methoxy, C-18-N-demethyl, 4,5-deoxy). Maytansinol and maytansinol analogues are described, for example, in U.S. Pat. No. 6,333,410. Maytansinol can be coupled using, e.g., an N-succinimidyl 3-(2-pyridyldithio)proprionate (also known as N-succinimidyl 4-(2-pyridyldithio)pentanoate or SPP), 4-succinimidyl-oxycarbonyl-a-(2-pyridyldithio)-toluene (SMPT), N-succinimidyl-3-(2-pyridyldithio)butyrate (SDPB), 2-iminothiolane, or S-acetylsuccinic anhydride.

### EXAMPLES

### Example 1

### IL-17RA-IL-17F Complex Expression and Crystallization

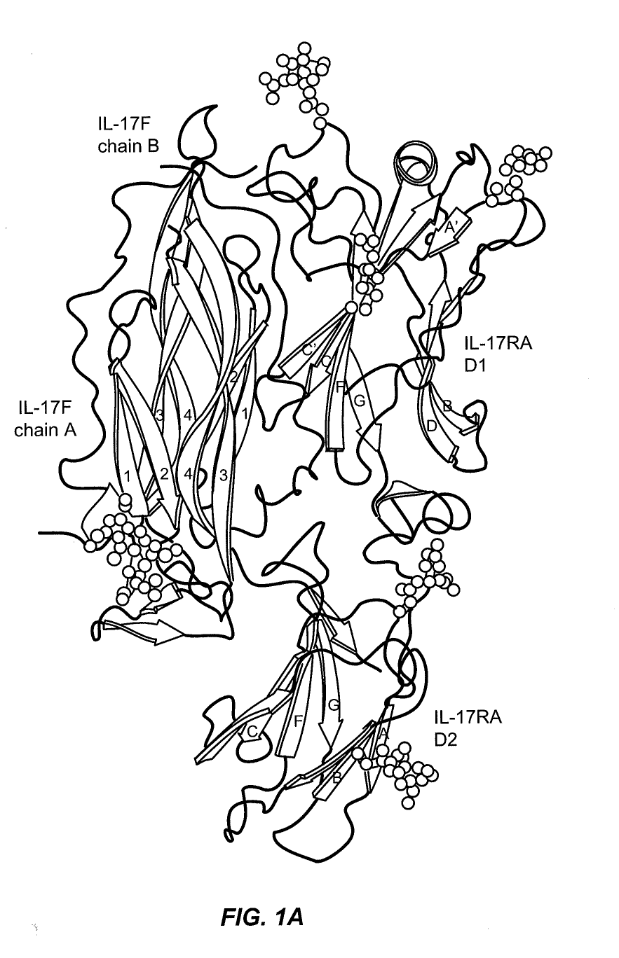

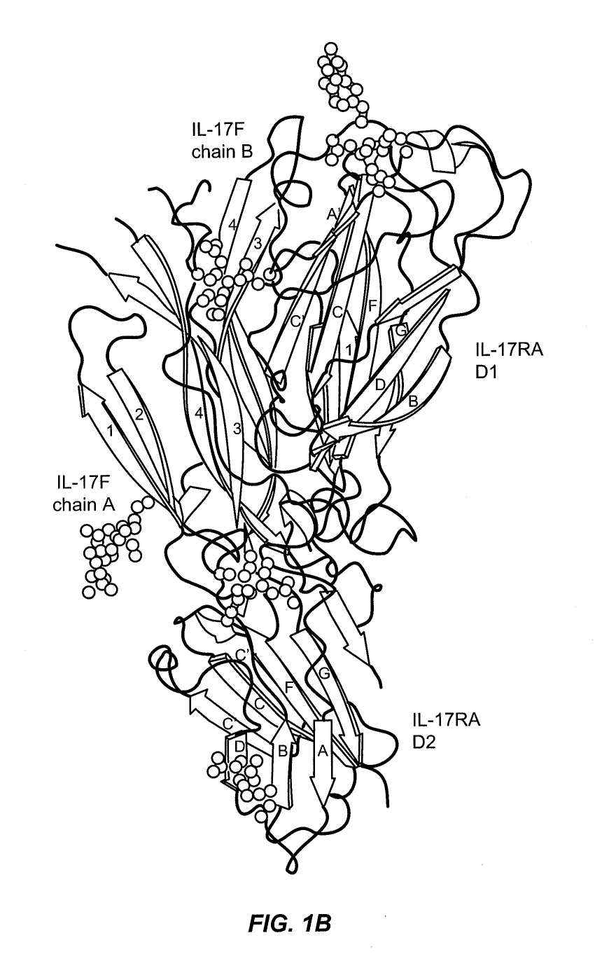

The crystal structure of IL-17RA bound to IL-17F at 3.3 Å resolution was determined using single isomorphous replacement with anomalous scattering (SIRAS) phasing. In order to generate the crystal, IL-17F was expressed and isolated from baculovirus. The IL-17RA extracellular domain (ECD) was produced using 293S GnTI-cells. To facilitate crystallization, the complex was methylated, and the heavily glycosylated receptor ECD was ‘shaved’ with endoglycosidase H prior to crystallization to improve homogeneity, leaving one GlcNAc residue at each of the Asn-linked glycosylation sites (FIG. 1). Biochemically the shaved and unshaved complexes behaved identically.

Applying gel filtration, mixtures of IL-17F or IL-17A with IL-17RA ECD resulted in co-elution of complexes with 2:2 (2 receptors+1 IL-17 dimer) and 1:2 (1 receptor+1 IL-17 dimer) stoichiometries, with the major species being the 1:2. The 2:2 was only detected at high protein concentrations, whereas at lower concentrations the 1:2 predominated even in the presence of excess IL-17RA. The crystals contained one IL-17RA bound to one IL-17F homodimer (FIG. 1). As discussed below, this ‘partial’ signaling complex may, in fact, be the biologically relevant form of the IL-17RA-IL-17F and IL-17RA-IL-17A complexes.

### Example 2

### IL-17RA-IL-17F Complex Overall Structure

The IL-17RA ectodomain is composed of two unusual FnIII domain modules joined by an 18-amino acid linker (FIG. 1). Although not apparent from the sequence, the IL-17RA structure is reminiscent of hematopoietic cytokine receptors in that it contains tandem b-sandwich domains; however, the domains themselves contain some substantial deviations from canonical FnIII folds, and the manner of ligand interaction is entirely distinct from other cytokine receptors. Residues 2-272 of the predicted 286 ectodomain residues (where residue 1 is the first amino acid of the mature peptide, as shown in SEQ ID NO:14) were modeled into continuous electron density for the receptor chain and five of the potential seven N-linked glycans were clearly visualized.

The first FnIII domain (D1) has an additional 40 amino acid N-terminal extension that forms a unique fold. The chain makes a hairpin-like turn bridged by a disulfide bond (Cys12-Cys19), and the second strand of the turn forms a β-strand (A′) that extends the FnIII n-sheet and then wraps around the face of the D1 domain, disulfide bonding with the C′ strand Cys95, before passing over the domain to start the A-strand of the FnIII domain. The interdomain linker region contains a short helix and is stabilized by an internal disulfide bond (Cys154-Cys165). The second FnIII domain (D2) has two atypical disulfide bonds, one linking the C-C′ loop (Cys214) to the D-F loop (Cys245) and a second within the F-G loop (Cys259-Cys263). A third disulfide bond is predicted to exist between F-G loop (Cys246) and C-terminus of the G-strand (Cys272), similar to that observed in class-II cytokine receptors, however this bond is not well defined in the current electron density map.

While the core structure of the IL-17RA-bound IL-17F molecule was essentially unchanged compared to that of the unliganded form of IL-17F, peripheral strands and loops underwent structural accommodations to facilitate binding to IL-17RA. The conformation observed in the unliganded IL-17F structure could not be maintained in the IL-17RA-bound state, as it would generate steric clashes with the N-terminal coil region of the receptor.

Each IL-17F monomer is composed of two pairs of anti-parallel β-sheets (strands 1-4) with the second and fourth strands connected by two disulfide bonds in a manner homologous to cysteine-knot family proteins. There is a 50 amino acid N-terminal extension of which residues 29-42 run parallel to strands 3 and 4 of the second IL-17F protomer. This coil region is stabilized by numerous interactions, including several hydrogen bonds with the adjacent strands.

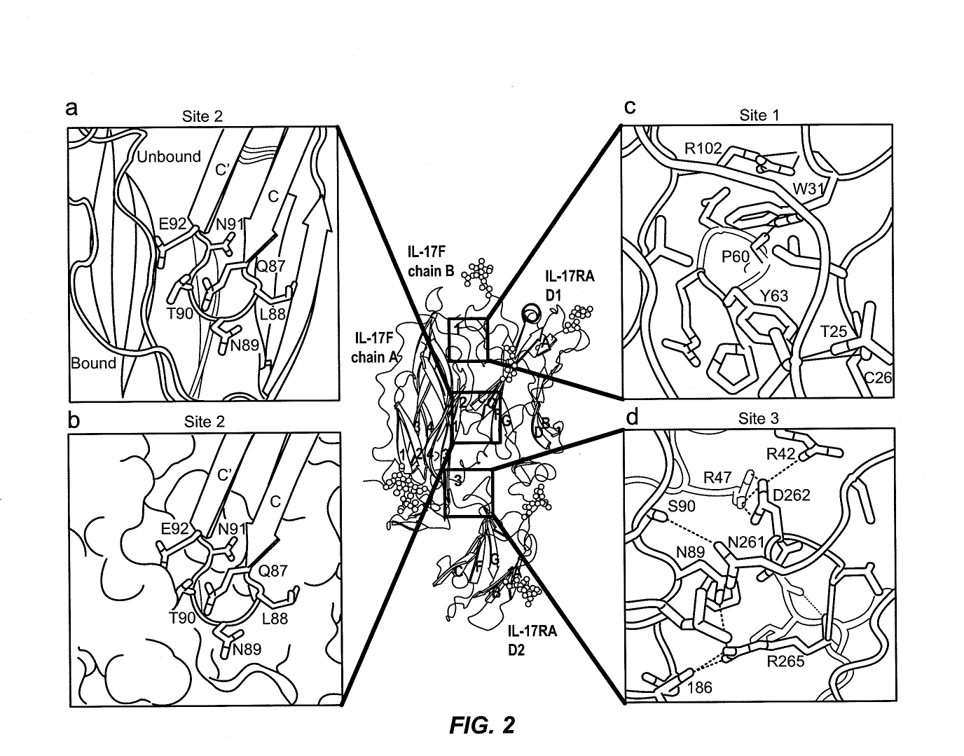

In the IL-17RA-bound IL-17F conformation this region (residues 33-42) moves out to open up the binding pocket and interact with the receptor (FIG. 2A). The first 24 amino acids of each IL-17F chain, and residues 105-109 from the 3-4 loop on one IL-17F protomer, could not be modeled. In the unliganded IL-17F structure Cys 17 forms a disulfide bond with Cys107 at the tip of the 3-4 loop on the adjacent IL-17F chain. These interchain disulfide bonds were not modeled, but were present as the protein behaved as a disulfide-linked dimer on SDS-PAGE.

### Example 3

### IL-17RA-IL-17F Binding Interface

The overall binding mode of IL-17F to IL-17RA, in which both receptor FnIII domains bind in a ‘side-on’ orientation and use edge strands to insert into a crevasse formed at the dimeric interface of the ligand, is unlike other cytokine or growth factor receptor complexes. IL-17RA forms an extensive binding interface with IL-17F, burying ˜2200 Å² of surface area; ˜70% of this buried surface area is mediated by the IL-17RA D1 domain. There are three major interaction sites at the binding interface (FIG. 2). Site 1 is formed between the N-terminal extension of IL-17RA (Thr25-Trp31 of SEQ ID NO:14) and the 1-2 loop (Pro60-Tyr63) plus the C-terminal region of strand 3 (Va1100, Arg102) of IL-17F chain B; this interaction buries ˜330 Å² (FIG. 2C). Trp31 of the receptor is buried in the center of this binding site; the main-chain O forms hydrogen bonds with Arg102 and the side chain forms hydrogen bonds with Pro60.

Two additional hydrogen bonds are formed between IL-17RA Thr25 and Cys26 and IL-17F Tyr63. Site 2 is the most prominent interface feature of the complex, and is composed of the IL-17RA D1 C′-C loop (Leu86-Arg93 of SEQ ID NO:14) which slots into a deep binding-pocket flanked by the N-terminal extension and strand 2 of IL-17F chain B and strand 3 of IL-17F chain A; this interaction buries almost 550 Å² (FIG. 2A, B).

This 8-amino acid IL-17RA loop forms extensive hydrophobic and polar interactions with both chains of IL-17F including a potential salt bridge between IL-17RA Glu92 and IL-17F chain B Arg37, and a hydrogen bond between the main-chain O of IL-17RA Asn89 and IL-17F chain A Asn95.

Site 3, which encompasses ˜410 Å² of buried surface area (BSA), is formed between the IL-17RA D2 F-G loop (Cys259-Arg265) and the C-terminal regions of stands 3 and 4 of IL-17F chain A, and the N-terminal extension of IL-17F chain B (FIG. 2D).

Site 3 is rich in charged interactions with nine potential hydrogen bonds and a salt bridge between IL-17RA Asp262 and IL-17F chain B Arg47. Overall the interface is extensive and is composed of numerous specific contacts. It is envisaged that an analogous binding mode will be used by other IL-17 receptor-cytokine pairs, given the sequence conservation of contact residues (discussed below). However, a greater bond-network and/or shape complementarity may be employed in the higher affinity complexes.

### Example 4

### Heterodimeric Receptor Complex Formation

The mechanism by which a homodimeric cytokine could possibly coordinate two different receptors was examined. Both IL-17RA and IL-17RC can bind independently to IL-17A and IL-17F, but both receptors are necessary for signaling. To further understand how the signaling complex is formed a surface plasmon resonance (SPR) strategy was devised using soluble proteins to measure the affinities of both the homodimeric and heteromeric receptor complexes for cytokine in vitro.

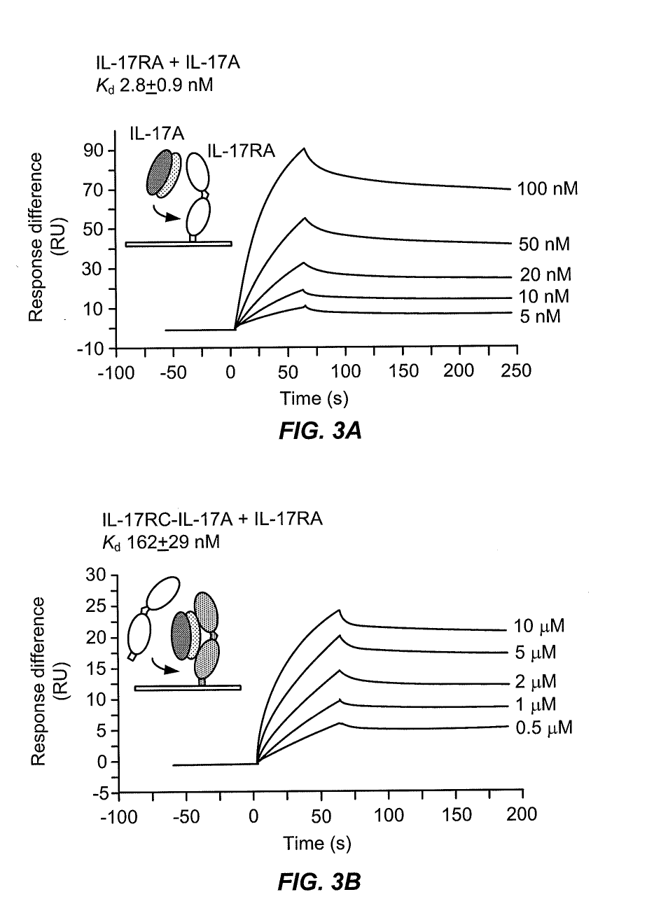

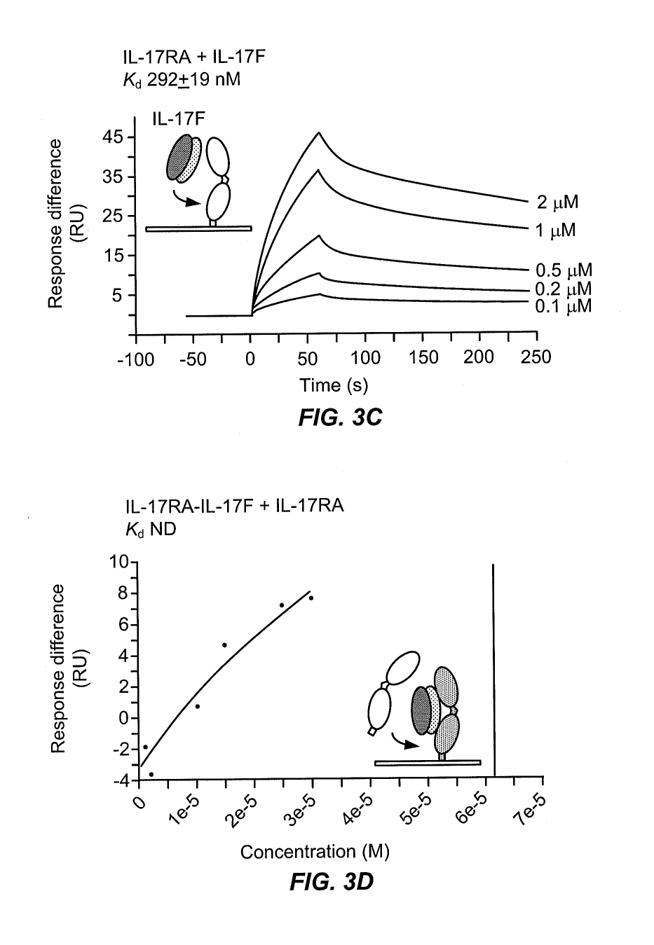

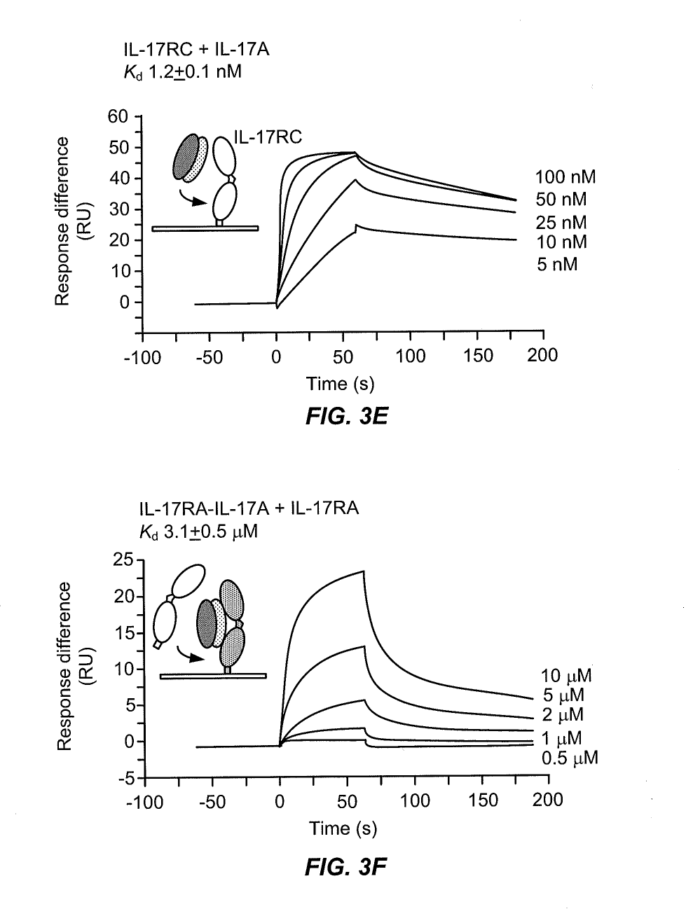

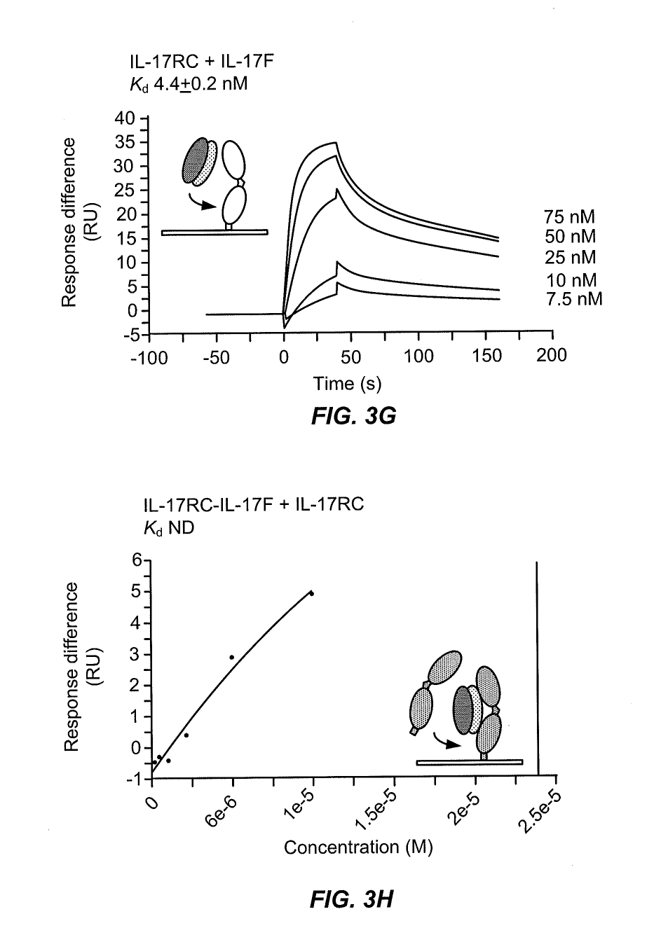

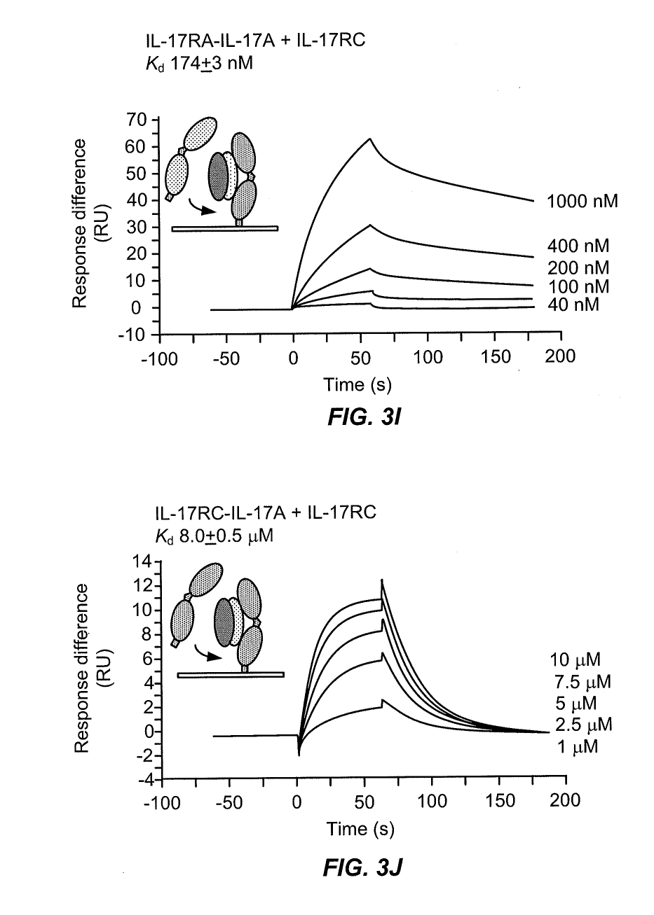

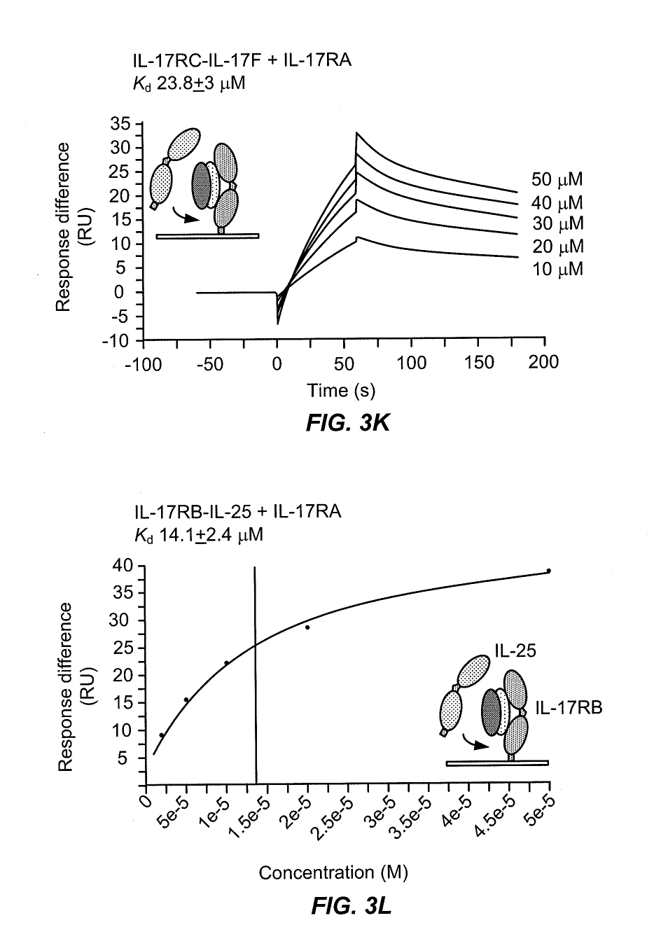

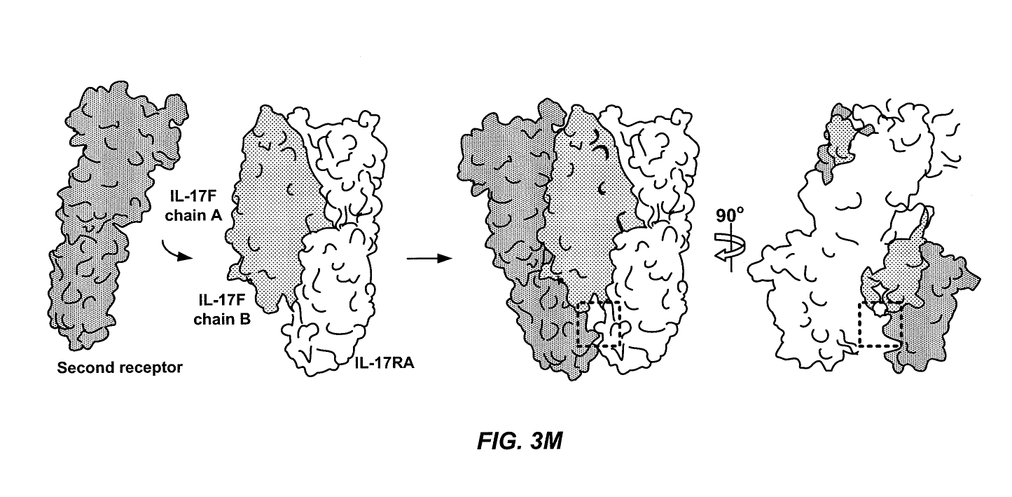

This strategy involved immobilizing one receptor on a SPR chip at a low coupling density in order to minimize possible homo-dimerization (e.g. cross-linking) of the receptors on the chip. The dimeric IL-17 cytokine was then captured by this receptor so that each receptor would be bound to one dimeric IL-17 ligand, leaving an exposed and accessible second receptor-binding site. The second receptor was subsequently passed over the preformed receptor-cytokine complexes to measure the affinity of the second receptor-binding event. In this fashion, the complex was assembled in a stepwise manner and each of the binding affinities was measured (FIG. 3).

IL-17A bound to both IL-17RA (2.8±0.9 nM) and IL-17RC (1.2±0.1 nM) with high affinity. Once IL-17A was bound by one IL-17RA molecule, the binding affinity for a second IL-17RA was reduced to 3.1±0.5 μM whereas the IL-17RC affinity for this second binding site was 174±3 nM. If the IL-17A was originally captured by IL-17RC, a second IL-17RA bound to the existing IL-17RC-IL-17A complex with 162±29 nM affinity; the binding affinity of a second IL-17RC to existing IL-17RC-IL-17A complex was only 8.0±0.5 μM.

A similar pattern was observed for IL-17F, which has a higher affinity for IL-17RC (4.4±0.2 nM) compared to IL-17RA (292±19 nM). Given the divergent affinities it seems likely that IL-17F would be initially captured by IL-17RC; once bound, the affinity of IL-17RA for the IL-17RC-IL-17F complex was 23.8±3

In contrast, the binding affinity of IL-17RA and IL-17RC for preformed IL-17RA-IL-17F and IL-17RC-IL-17F complexes, respectively, was so weak that it could not be accurately calculated over the concentration range used for these experiments.

Thus, these findings clearly show that engagement of IL-17RA or IL-17RC by IL-17A or IL-17F encourages a preference for the second receptor-binding site to engage a different receptor and thereby to form the heterodimeric receptor complex.

IL-17RA has been implicated in IL-17E (also known as IL-25) signaling together with IL-17RB. IL-25 promotes Th2 inflammatory responses and shares approximately ˜20% identity with IL-17A and IL-17F. Binding experiments have demonstrated that whilst IL-25 binds to IL-17RB with high affinity, it has no apparent affinity for IL-17RA.

To examiner whether IL-17RA may only specifically bind IL-25 once IL-25 is captured by IL-17RB, IL-17RB was immobilized on an SPR chip, IL-25 was passed over this chip and the affinity of IL-17RA for the IL-17RB-IL-25 complex was measured. IL-17RA was observed to bind to the IL-17RB-IL-25 complex with 14.1±2.4 μM affinity (FIG. 3). At concentrations up to 50 μM, no interaction could be observed between IL-17RA and IL-25, or between the IL-17RB-IL-25 complex and a second IL-17RB molecule. Together with the IL-17A and IL-17F binding data, these results indicate that the formation of the heteromeric complex may be mediated by allostery and/or an interaction between the receptors.

This concept was further addressed by modeling a second IL-17RA molecule to form the hypothetical 2:2 receptor-cytokine complex (FIG. 3B). Assuming that the second receptor binds in an identical fashion to the first, the base of IL-17RA D2 would come into very close proximity with the D2 of the second IL-17RA (FIG. 3B, dashed box). In the case of two IL-17RA molecules bound to IL-17F, His212 on the C-C′ loop of one IL-17RA would clash with the second IL-17RA His212. This potential interaction site may allow the receptors to regulate their pairing.

Steric clashes may cause reduced affinity for a second identical receptor, or favorable receptor-receptor interactions may stabilize heteromeric receptor complexes. The data suggests that receptor heterodimers will likely be the predominant signaling species.

### Example 5

### IL-17RA Functions as a Common Receptor

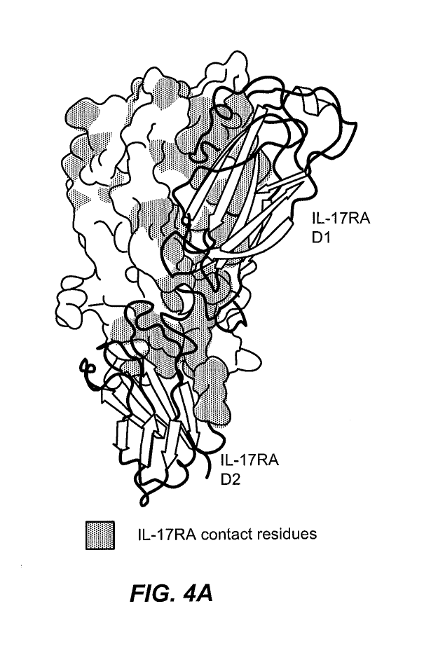

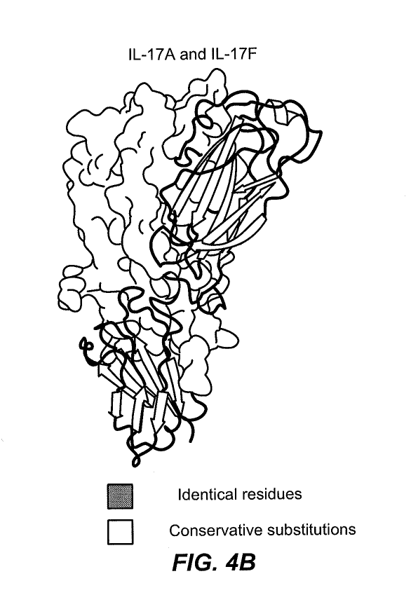

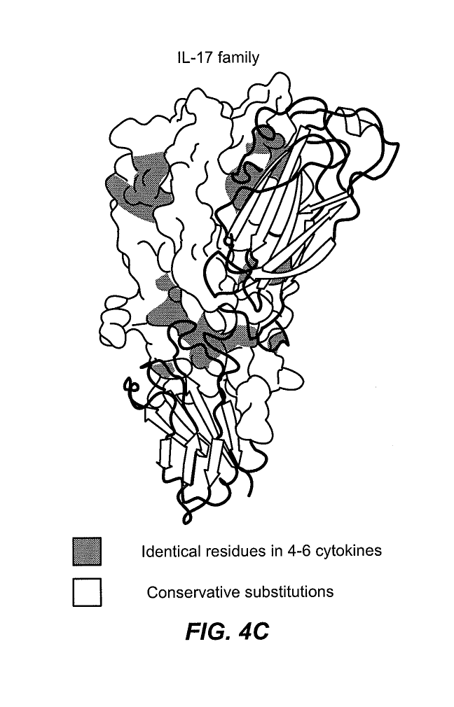

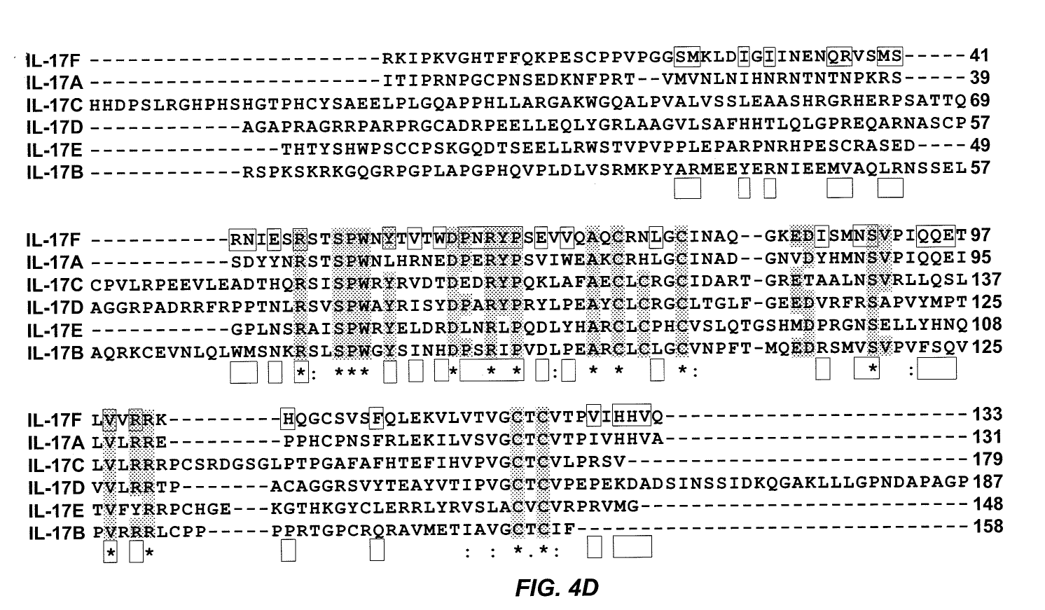

IL-17RA binds to IL-17A with ˜100-fold higher affinity than IL-17F. IL-17A and IL-17F share ˜50% identity, and mapping the conserved residues onto the structure of IL-17F reveals a horseshoe-shaped ring of variable residues around the receptor-binding pocket (FIG. 4). The majority of the IL-17RA C′-C loop interactions are formed with residues that differ between the IL-17A and IL-17F molecules whereas the N-terminal region and IL-17RA D2 F-G loop interactions involve predominately conserved residues.

The extracellular region of IL-17RA can also bind to the IL-17RB-IL-25 complex, and it was recently shown that IL-17RD can interact with IL-17RA to mediate IL-17A signaling. Given this association of IL-17RA with diverse IL-17 family members, the IL-17RA may act as a shared receptor analogous to those utilized in class I cytokine receptor complexes.

To investigate this possibility, the residues conserved among all IL-17 family members were mapped onto the IL-17F surface. Analyzing the location of these residues in the IL-17RA-IL-17F complex, it seems plausible that IL-17RA contacts these conserved residues with the N-terminal region of the D1 domain and the F-G loop of the D2 domain (FIG. 4C).

In contrast, IL-17RA may modulate specificity for each cytokine by contacting non-conserved cytokine residues with the C-C′ loop (FIG. 4C). Collectively, then, IL-17RA appears to use a strategy of cross-reactivity based on a subset of conserved contacts, amongst a background of distinct contacts, with several different IL-17 cytokines.

### Example 6

### Receptor Binding Modes of Cysteine-Knot Growth Factors

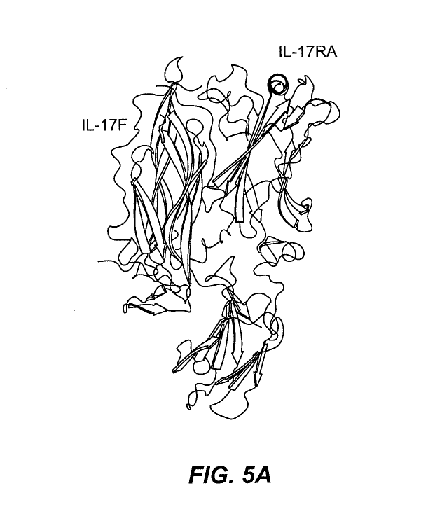

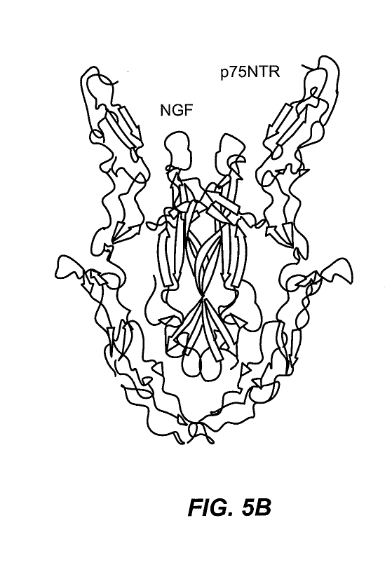

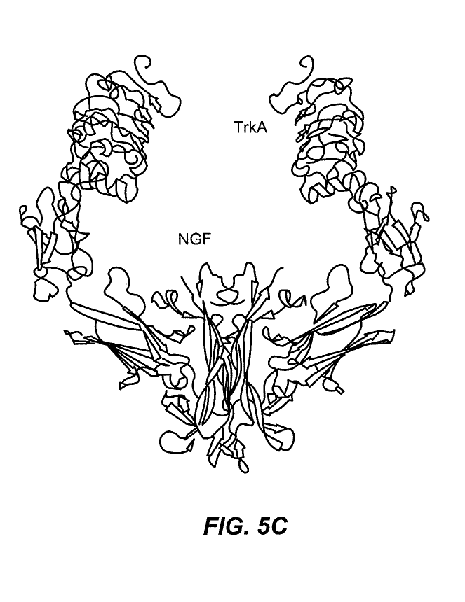

Several crystal structures for receptor-cysteine-knot growth factor ligand complexes, such as nerve growth factor (NGF), vascular endothelial growth factor (VEGF) two glial cell-derived neurotrophic factor (GDNF) family members, and others; these structures can serve as instructive comparisons with the mode of ligand engagement mediated by IL-17RA (FIG. 5). In the complex of NGF bound to the p75 neurotrophin receptor (p75NTR, a death receptor family member), the receptor bears no structural similarity to IL-17RA; however, like IL-17RA, p75NTR engages NGF within a concave groove at the ligand dimer interface (FIG. 5B).

In the TrkA complex with NGF, an immunoglobulin (Ig)-domain in TrkA, which is structurally related to the FnIII domains of IL-17RA, is used for ligand binding. However, the Ig-domain of TrkA binds end-on to a flat face in the ‘saddle’ of NGF formed by the NGF n-sheets; thus the mode of binding is distinct (FIG. 5C). The NGF-p75NTR complex has been reported as both 1:2 and 2:2 complexes that may represent partial and complete forms of a homodimeric p75 signaling complex, respectively (28, 30). However, in that case, homodimeric NGF ligand engages two identical p75 molecules, and thus does not require a structural mechanism for the symmetric dimeric ligand to heterodimerize two different receptors.

### Example 7

### Human IL-17RC or Human IL-17RA Binding

Binding of Biotinylated Cytokines to Transfected Cells.

Baby Hamster Kidney (BHK) cells transfected with expression vectors encoding human IL-17RA, human IL-17RC, or both of these receptors are assessed for their ability to bind biotinylated human IL-17A, human IL-17F, and their variants including antagonists described herein. Cells are harvested with Versene™, counted and diluted to 10⁷ cells per ml in staining media (SM), which is HBSS plus 1 mg/ml bovine serum albumin (BSA), 10 mM HEPES, and 0.1% sodium azide (w/v). Biotinylated human IL-17A, human IL-17F, and other proteins of interest are incubated with the cells on ice for 30 minutes at various concentrations. After 30 minutes, excess protein is washed away with SM and the cells are incubated with a 1:100 dilution of streptavidin conjugated to phycoerythrin (SA-PE) for 30 minutes on ice. Excess SA-PE is washed away and cells are analyzed by flow cytometry. The amount of binding is quantitated from the mean fluorescence intensity of the staining.

Binding of Biotinylated Cytokines to Human Peripheral Blood Mononuclear Cells (PBMC).

PBMCs are prepared from whole blood by Ficoll™ density gradient centrifugation. PBMC at 10⁷ cells per ml are simultaneously incubated with biotinylated IL-17A or IL-17F or proteins of interest at 1 μg/ml and fluorochrome conjugated antibodies to specific cell surface proteins that are designed to distinguish various white blood cell lineages. These markers include CD4, CD8, CD19, CD11b, CD56 and CD16. Excess antibody and cytokine are washed away, and specific cytokine binding is detected by incubating with SA-PE as described above. Samples are analyzed by flow cytometry.

Inhibition of Specific Binding.

Binding studies are performed as discussed above, but excess unlabeled human IL-17A and IL-17F or excess unlabeled proteins of interest such as proteins described herein are included in the binding reaction. In studies with BHK cells, the amount of unlabeled protein is varied over a range of concentrations and unlabeled IL-17A and proteins of interest are evaluated for ability to compete for binding of both IL-17A and IL-17F to both IL-17RC and IL-17RA.

### Example 8

### Murine NIH3T3 Cells Respond to Human IL-17A and IL-17F

Murine NIH3T3 cells are transfected with Kz142 adenovirus particles containing a consensus NF-κB binding site, the tandem NF-κB binding sites of the human immunodeficiency virus-1 long terminal repeat, two copies of the collagenase AP-1 element, and a single copy of the c-Jun TRE ligated into a luciferase reporter cassette and placed in the pACCMV.pLpA adenoviral shuttle vector as described in Blumberg et al. (2001) Cell 104:9-19.

Following the overnight incubation with the adenovirus particle reporter, treatments (e.g., with IL-17A, IL-17F, or others proteins of interest) are prepared in serum free media containing 0.28% BSA. The adenovirus particles and media are removed and the appropriate doses are given. Incubation at 37° C. and 5% CO₂ is continued for 4 hours, after which the media is removed, cells lysed for 15 minutes and mean fluorescence intensity (MFI) measured using the luciferase assay system and reagents. (Promega, Madison, Wis.) and a microplate luminometer. Stable cell lines can also be made. Stable and/or transient cell lines can be used to evaluate a protein described herein for activity.

### Example 9

### IL-17A Induces Elevated Levels of IFNγ and TNFα in Human Peripheral Blood Mononuclear Cells

Human peripheral blood mononuclear cells (PBMC) are purified by Ficoll™ density gradient centrifugation and then incubated overnight at 37° C. in media alone, 50 ng/ml anti-human CD3 antibody, or the combination of 50 ng/ml anti-human CD3 antibody plus 1 μg/ml anti-human CD28 antibody. Replicate cultures for each of these conditions are set up and are given no cytokine, 25 ng/ml human IL-17A, 25 ng/ml human IL-17F, or varying concentrations of a protein of interest (for example in the presence of cytokine). After 24-hour incubations, supernatants from each culture are harvested and assayed for cytokine content using B-D Bioscience's human Th1/Th2 Cytometric Bead Array (CBA). Cultures stimulated with anti-CD3 or anti-CD3 plus anti-CD28 and supplemented with IL-17A contained significantly elevated levels of IFNγ and TNFα. Proteins of interest can be evaluated for their ability to inhibit IL-17A induction of IFNγ and TNFα.

### Example 10

### Mouse Collagen Induced Arthritis (CIA) Model

The mouse Collagen Induced Arthritis (CIA) model can be used to evaluate therapeutic potential of drugs (such as proteins described herein) to treat human arthritis. Eight to ten-week old male DBA/IJ mice (Jackson Labs; 25-30 g each) are used for these studies. On day-21, animals are given an intradermal tail injection of 100 μL of 1 mg/ml chick type II collagen formulated in Complete Freund's Adjuvant and three weeks later on Day 0 mice are given the same injection except prepared in Incomplete Freund's Adjuvant. Animals begin to show symptoms of arthritis following the second collagen injection, with most animals developing inflammation within 1-2 weeks. The extent of disease is evaluated in each paw by using a caliper to measure paw thickness, and by assigning a clinical score (0-3) to each paw: 0=Normal, 0.5=Toe(s) inflamed, 1=Mild paw inflammation, 2=Moderate paw inflammation, and 3=Severe paw inflammation as detailed below.

Incidence of disease in this model is typically 95-100%, and 0-2 non-responders (determined after 6 weeks of observation) are typically seen in a study using 40 animals. Note that as inflammation begins, a common transient occurrence of variable low-grade paw or toe inflammation can occur. For this reason, an animal is not considered to have established disease until marked, persistent paw swelling has developed.

All animals are observed daily to assess the status of the disease in their paws, which is done by assigning a qualitative clinical score to each of the paws. Every day, each animal has its four paws scored according to its state of clinical disease. To determine the clinical score, the paw is thought of as having three zones, the toes, the paw itself (manus or pes), and the wrist or ankle joint. The extent and severity of the inflammation relative to these zones is noted including: observation of each toe for swelling; torn nails or redness of toes; notation of any evidence of edema or redness in any of the paws; notation of any loss of fine anatomic demarcation of tendons or bones; evaluation of the wrist or ankle for any edema or redness; and notation if the inflammation extends proximally up the leg. A paw score of 1, 2, or 3 is based first on the overall impression of severity, and second on how many zones are involved.

Treatments: Established disease is defined as a qualitative score of paw inflammation ranking 1 or more. Once established disease is present, the date is recorded, designated as that animal's first day with “established disease”, and treatment started. Mice are treated with PBS, or with varying doses of the protein of interest, i.p. every other day for a total of five doses: 150 μg; 75 μg; 25 μg; and 10 μg.

Blood is collected throughout the experimental period to monitor serum levels of anti-collagen antibodies, as well as serum immunoglobulin and cytokine levels. Animals are euthanized 48 hours following their last (5th) treatment, about 10 days following disease onset. Blood is collected for serum, and all paws are collected into 10% NBF for histology. Serum is collected and frozen at −80° C. for immunoglobulin and cytokine assays. A dose-dependent, significant reduction in clinical score severity in treated mice indicates a biological effect for the protein in this test system.

### Example 11

### Additional Disease Model

The Inflammatory Bowel Disease (IBD) model is designed to show that cultured intestinal tissue from patients with IBD produce higher levels of inflammatory mediators compared to tissue from healthy controls. This enhanced production of inflammatory mediators (including but not limited to IL-1β, IL-4, IL-5, IL-6, IL-8, IL-12, IL-13, IL-15, IL-17 A and F, IL-18, IL-23, TNF-α, IFN-γ, MIP family members, MCP-1, G- and GM-CSF, etc.) contributes to the symptoms and pathology associated with IBDs such as Crohn's disease (CD) and ulcerative colitis (UC) by way of their effect(s) on activating inflammatory pathways and downstream effector cells. These pathways and components then lead to tissue and cell damage/destruction observed in vivo. Therefore, this model can simulate this enhanced inflammatory mediator aspect of IBD. Furthermore, when intestinal tissue from healthy controls or from human intestinal epithelial cell (IEC) lines is cultured in the presence of these inflammatory components, inflammatory pathway signaling can be observed, as well as evidence of tissue and cell damage.

Therapeutics that would be efficacious in human IBD in vivo would work in the above ex vivo or IEC models by inhibiting and/or neutralizing the production and/or presence of inflammatory mediators.

In this model, human intestinal tissue is collected from patients with IBD or from healthy controls undergoing intestinal biopsy, re-sectioning or from post-mortem tissue collection, and processed using a modification of Alexakis et al. (Gut 53:85-90; 2004). Under aseptic conditions, samples are gently cleaned with copious amounts of PBS, followed by culturing of minced sections of tissue, in the presence of complete tissue culture media (plus antibiotics to prevent bacterial overgrowth).

Samples from the same pool of minced tissue are treated with one of the following: vehicle (PBS); recombinant human (rh) IL-17A; rhIL-17F; or rhIL-17A+rhIL-17F. In addition, these samples can be treated with or without an antagonist of either IL-17A, IL-17F, IL-17B, IL-17C, IL-17D, and IL-17E alone or in combination. This experimental protocol is followed for studies with human IEC lines, with the exception that cells are passaged from existing stocks. After varying times in culture (from 1 h to several days), supernatants are collected and analyzed for levels of inflammatory mediators, including those listed above. In samples from patients with IBD or in samples treated with rhIL-17A and/or F, levels of inflammatory cytokines and chemokines are elevated compared to untreated healthy control tissue samples. Proteins of interest can be evaluated for ability to reduce the production of inflammatory mediators, and thus, to be efficacious in human IBD.

Proteins of interest can be evaluated in a mouse model for dry eye disease. Dry eye can be induced in mice by subcutaneous injection of scopolamine and then placement of the mice in controlled-environment chambers. The controlled environment chamber can be controlled for relative humidity, temperature, and air flow. See, e.g., Barabino et al., Invest. Ophth. Vis. Sci., 46:2766-71, 2005. Various mouse strains can be used. These include, e.g., C57BL/6, BALB/c, NZB/W, and MLR/lpr, MLR/+. Other animals, e.g., rabbits, rats, monkeys, dogs, and cats, can also be used as dry eye disease models. See e.g., Nguyen and Peck, Ocul. Surf., 7(1):11-27, 2009 (including Table 1), and Barabino and Dana, Invest. Ophth. Vis. Sci., 45(6): 1641-46, 2004.

By way of example, dry eye can be induced in normal healthy 6 to 10 weeks old female C57BL/6 mice by continuous exposure to dry environment in a controlled environmental chamber with humidity less than 30% (generally about 19%), high airflow (generally greater than about 15 liters/minute) and constant temperature (about 22° C.). The mice placed in the chamber are also treated with scopolamine to inhibit tear secretion. One quarter of a sustained release transdermal scopolamine patch (Novartis, Summit N.J.) is applied to the depilated mid-tail of mice every 48 hours, or the scopolamine can be injected, e.g., 750 μg, twice daily subcutaneously. The combination of the controlled environmental chamber and scopolamine produces severe dry eye in a relative short timeframe (about 2-4 days). Mice can be treated after disease onset with a protein of interest for 7 to 14 days under these conditions and compared to placebo or vehicle treated controls. Mice can be monitored and evaluated for dry eye, e.g., by performing: (a) an assessment of aqueous tear production; (b) corneal fluorescein staining which is a marker of corneal surface damage; (c) an assessment of goblet cell density in the superior and inferior conjunctiva; (d) general ophthalmic examination, e.g., for conjunctival epithelial morphology; (e) scanning electron microscope examination of corneal surface; and (f) immunohistochemistry.

### Example 12

### Rheumatoid Arthritis (RA) and Osteoarthritis (OA) Model

This model is designed to show that human synovial cultures (including synovial macrophages, synovial fibroblasts, and articular chondrocytes) and explants from patients with RA and OA produce higher levels of inflammatory mediators compared to cultures/explants from healthy controls. This enhanced production of inflammatory mediators (including but not limited to oncostatin M, IL-1β, IL-6, IL-8, IL-12, IL-15, IL-17 A and F, IL-18, IL-23, TNF-α, IFN-γ, IP-10, RANTES, RANKL, MIP family members, MCP-1, G- and GM-CSF, nitric oxide, etc.) contributes to the symptoms and pathology associated with RA and OA by way of their effect(s) on activating inflammatory pathways and downstream effector cells.

These pathways and components then lead to inflammatory infiltrates, cartilage and matrix loss/destruction, bone loss, and upregulation of prostaglandins and cyclooxygenases. Therefore, this model can simulate the destructive inflammatory aspects of RA and OA in in vitro and ex vivo experiments. Furthermore, when explants and synovial cultures from healthy controls are cultured in the presence of several of these inflammatory components (e.g. oncostatin M, TNF-α, IL-1β, IL-6, IL-17A and F, IL-15, etc.), inflammatory pathway signaling can be observed. Therapeutics that would be efficacious in human RA in vivo may have an effect in the above in vitro and ex vivo models by inhibiting and/or neutralizing the production and/or presence of inflammatory mediators.

In this model, human synovial explants are collected from patients with RA, OA, or from healthy controls undergoing joint replacement or from post-mortem tissue collection, and processed using a modification of Wooley and Tetlow (Arthritis Res 2: 65-70, 2000) and van't H of et al. (Rheumatology 39:1004-1008, 2000). Cultures of synovial fibroblasts, synovial macrophages and articular chondrocytes are also studied. Replicate samples are treated with one of the following: vehicle (PBS); recombinant human (rh) IL-17A; rhIL-17F; or rhIL-17A+rhIL-17F, and some samples contain various combinations of oncostatin M, TNF-α, IL-1, IL-6, IL-17A, IL-17F, and IL-15. In addition, these can be evaluated in the presence or absence of a protein of interest. After varying time of culture (from 1 h to several days), supernatants are collected and analyzed for levels of inflammatory mediators, including those listed above. In samples from patients with RA or OA, or in samples treated with rhIL-17A and/or F (either alone or in combination with other inflammatory cytokines), levels of inflammatory cytokines and chemokines are elevated compared to untreated healthy control explants or in untreated cell cultures. Proteins of interest can be evaluated for ability to reduce the production of inflammatory mediators, and thus, to be efficacious in human RA and OA.

### Example 13

### Induction of G-CSF, IL-6 and IL-8

Human small airway epithelial cells (SAEC) treated with human IL-17A or with human IL-17F can show a dose-dependent induction of G-CSF, IL-6, and IL-8, e.g., by evaluation of cell supernatants 48 hr after treatment. Proteins of interest can be evaluated for their ability to inhibit this induction.

### Example 14

### Human Rheumatoid Arthritis (“RA”) and Osteoarthritis (“OA”) Samples

These models are designed to show that human synovial cultures (including synovial macrophages, synovial fibroblasts, and articular chondrocytes) and explants from patients with RA and OA produce higher levels of inflammatory mediators compared to cultures/explants from healthy controls, which in turn can contribute to the degradation of extracellular matrix components (e.g. bone, cartilage, etc), which is a hallmark of these diseases. In addition, the co-culture models described below are designed to show that inflammatory mediators present in RA/OA synovial fluid and/or activated T cells can also result in greater inflammation and matrix degradation.

The enhanced production of inflammatory mediators (including but not limited to oncostatin M, IL-1β, IL-6, IL-8, IL-12, IL-15, IL-17 A and F, IL-18, IL-23, TNF-α, IFN-γ, IP-10, RANTES, RANKL, MIP family members, MCP-1, MMP-9, G- and GM-CSF, nitric oxide, etc.) contributes to the symptoms and pathology associated with RA and OA by way of their effect(s) on activating inflammatory pathways and downstream effector cells. These pathways and components then lead to inflammatory infiltrates, cartilage and matrix loss/destruction, bone loss, and upregulation of matrix metalloproteases, prostaglandins and cyclooxygenases. Therefore, these models can simulate the destructive inflammatory aspects of RA and OA in in vitro and ex vivo experiments.

Furthermore, when explants and synovial cultures from healthy controls are cultured in the presence of exogenously added inflammatory components (e.g. oncostatin M, TNF-α, IL-1β, IL-6, IL-17A and F, IL-15, etc.), or alternatively, in the presence of synovial fluid from RA patients (which would contain inflammatory components endogenously), inflammatory and degradative pathway signaling can be observed. Therapeutics that would be efficacious in human RA in vivo would work in the above in vitro and ex vivo models by inhibiting and/or neutralizing the production and/or presence of inflammatory mediators.

In these models, human synovial explants are collected from patients with RA, OA, or from healthy controls undergoing joint replacement or from post-mortem tissue collection, and processed using a modification of Wooley and Tetlow (Arthritis Res 2: 65-70; 2000) and van't H of et al. (Rheumatotogy 39:1004-1008; 2000). Cultures of synovial fibroblasts, synovial macrophages and articular chondrocytes are also studied. Replicate samples are treated with one of the following: vehicle (PBS); recombinant human (rh) IL-17A; rhIL-17F; or rhIL-17A+rhIL-17F, and some samples contain various combinations of oncostatin M, TNF-α, IL-1, IL-6, IL-17A, IL-17F, and IL-15. A separate set of samples is treated with activated human T cells, or synovial fluid from healthy controls or patients with RA or OA.

After varying time of culture (from 1 h to several days), supernatants and cells are collected and analyzed for levels of inflammatory mediators and cartilage/bone/matrix biomarkers, including those listed above. Samples can be treated with a protein of interest and evaluated for ability to reduce the production of inflammatory and cartilage/bone/matrix degradative mediators, and thus, to be efficacious in human RA and OA.

### Example 15

### Single Chain Human IL17A:IL17F Heterodimers

Recombinant human IL17A:IL17F heterodimer protein or recombinant IL17A:IL17F-variant is produced from expression of the appropriate single chain construct in CHO DXB11 cells and cell culture in a WAVE apparatus. One construct is comprised of sequences for human IL-17A at the N-terminus with IL-17F at the C-terminus linked with a (G₄S)₃ linker; another exemplary construct is comprised of sequences for human IL-17A at the N-terminus with a IL-17F-variant at the C-terminus linked with a (G₄S)₃ linker. A His tag can be included at the C-terminus for product capture.

An exemplary purification method is described in US 20080241138. Briefly, it can include an acid precipitation step, filtration, followed by chromatography. For example, approximately 10 L of conditioned media are harvested and sterile filtered using a 0.2 μm filter. The media is adjusted to pH 5.0 with addition of acetic acid while stirring. After precipitation, the pH-adjusted media is again filtered through a two stage 0.8 to 0.2 micron filter. The media can then be subjected to cation exchange chromatography on SP Fast Flow resin, and eluted with a salt gradient. Peak fractions can then be subjected to IMAC chromatography, e.g., using a 5 mL HISTRAP® IMAC column (GE Healthcare). After elution with imidazole, peak fractions can be subjected size exclusion chromatography, e.g., on SUPERDEX® 200. Peak fractions can then be pooled and used. Fractions can be evaluated by Western analysis (e.g., with an anti-His tag antibody) and/or by SDS-PAGE with Coomassie gel staining.

### Example 16

### Expression and Purification of IL-17RA and IL-17F

The native signal peptide and extracellular region of human IL-17RA (residues 1-286) was cloned into the BacMam® expression vector pVLAD637. Recombinant protein was transiently expressed in suspension 293 GnTI-cells grown in Pro293™ media (Lonza) supplemented with 1% fetal calf serum (FBS) and 10 mM Na butyrate at 37° C. Full length IL-17F with a C-terminal hexa-His tag was cloned into the pAcGP67-A expression vector (BD Biosciences) and the protein secreted by High Five insect cells grown in Insect Xpress™ media (Lonza) at 27° C. The supernatants containing the IL-17RA and IL-17F proteins were mixed and concentrated before Ni-affinity purification. The IL-17RA protein was deglycosylated via endoglycosidase-H treatment and the IL-17RA and IL-17F purification tags cleaved using 3C-protease and carboxypeptidase A (Sigma-Aldrich).

The protein complex was subjected to reductive lysine methylation using dimethylamine-borane complex and formaldehyde as described by Walter et al. The IL-17RA-IL-17F complex was further purified using a Superdex® 200 size exclusion column (GE Healthcare) equilibrated in 10 mM Hepes pH 7.4 and 150 mM NaCl. Fractions containing the IL-17RA-IL-17F complex were concentrated to ˜15 mg/ml for crystallization trials.

Seleno-methionine (SeMet) labeled IL-17RA protein was prepared as described. Untransfected adherent 293 GnTI-cells were cultivated in FBS-supplemented DMEM media (Invitrogen). The media was exchanged after a single phosphate-buffered saline wash, for Met and Cys-free DMEM (Invitrogen) supplemented with 40 mg/l L-Cys, 45 mg/l selenon-L-Met, 2% FBS, L-glutamate, Na pyruvate, IL-17RA BacMam virus and 10 mM Na butyrate. Expression was allowed to proceed for 72 hours. IL-17RA-SeMet protein supernatants were mixed with IL-17F and purified as described above.

For binding experiments, proteins were expressed and purified essentially as described above. The IL-17RA, IL-17RB and IL-17RC extracellular domains were expressed by 293s GnTI-cells with and without a C-terminal BirA ligase tag. IL-17RC was expressed with an additional C-terminal Fc tag that was cleaved by 3C-protease prior to size exclusion chromatography. IL-17A, IL-17F and IL-25 cytokines were expressed by High Five cells with C-terminal hexa-His tags. Proteins were enzymatically biotinylated using BirA ligase and purified via size exclusion chromatography.

### Example 17

### Crystallization and X-Ray Data Collection

IL-17RA-IL-17F complexes were initially grown via hanging-drop vapor diffusion in 10% PEG6000 and 0.1 M bicine pH 9.0. Optimized native and SeMet protein complex crystals were grown in PEG6000 (4-14%) and 0.1 M CAPSO buffer (pH 9.1-9.3) with 20 mM CaCl2 or 10 mM CaCl2 and 1.5% w/v trimethylamine N-oxide dihydrate added directly to the protein-precipitant drop. Heavy metal derivatives were prepared by soaking the crystals in well solution supplemented with 0.5 mM K2PtCl4 and 2% ethylene glycol for 6 hours. Crystals were cryo-protected prior to data collection in the well solution plus 20-25% ethylene glycol and cooled to 100 K.

The crystals belong to the space group P41212 and have unit cells dimensions of ˜171, 171, 83 Å. The initial native data set was collected at Stanford Synchrotron Radiation Lightsource (SSRL) beamline 9-2 (Stanford, Calif.). The Pt-derivative and SeMet datasets were collected at SSRL beamline 11-1. The higher resolution native dataset was collected at the Advanced Photon Source (APS) beamline ID-23D (Argonne, Ill.). All data was indexed and integrated using the program Mosflm40 and scaled with SCALA from the CCP4 suite. The diffraction is anisotropic and the initial native dataset was also subjected to ellipsoidal truncation and anisotropic scaling using the diffraction anisotropy server rendering a data set scaled to 3.4, 3.4 and 3.9 Å.

### Example 18

### Structure Determination and Refinement

A molecular replacement solution for a single IL-17F homodimer was determined using the program Phaser43 with the previously determined 2.85 Å IL-17F structure as a model (PDB ID 1 JPY). The initial maps showed additional density on one side of the IL-17F dimer illuminating the binding site for IL-17RA. Phases were calculated using a K₂PtCl₄ derivative via single isomorphous replacement with anomalous scattering (SIRAS) in the program Sharp. Density modified maps were calculated assuming 71% solvent and including the partial model from the IL-17F molecular replacement for 10 out of 20 rounds. A partial model of the IL-17RA main chain was manually built into this map using the program Coot.

The position of the IL-17RA Met residues was calculated via fast Fourier transform (FFT) to generate an anomalous difference map using the program FFT in the CCP4 suite. As the SeMet dataset was not isomorphous with the native dataset and the signal too weak to locate the sites via single anomalous difference (SAD) phasing methods, the partially built model was used as a molecular replacement model for the SeMet dataset and the calculated phases used to find the selenonium peaks.

Three of a potential six SeMet residues were located, corresponding to IL-17RA Met159, Met166 and Met218. These Met positions, in addition to the predicted Asn-linked glycosylation sites and disulfide bonds were used to register the polypeptide in the density and complete building the initial IL-17RA model. Iterative rounds of coordinate and B-factor refinement were performed using the program Phenix46 intersected with manual model building in Coot. Initial rounds of model building utilized B-factor sharpened σ_{A}-weighted phased-combined maps calculated by the program CNS.

The final model was refined to 3.3 Å with an R_{factor} and R_{free} of 22.7% and 25.3% respectively. There is one IL-17RA-IL-17F complex in the asymmetric unit. The model includes a dimethyl-lysine at position 43 of the IL-17RA chain, five single N-Acetylglucosamine (GlcNAc) sites on the IL-17RA chain, one site with two GlcNAc residues on the IL-17F chain B and a calcium ion. The programs PROCHECK48 and WHAT_CHECK were used to assess the geometry of the final model. The CCP4 suite programs Contact and Areaimol were used to determined the interface contacts and buried surface area respectively. All structural figures were generated using the program Pymol.

### Example 19

### Affinity Measurements

Binding affinities were calculated via surface plasmon resonance (SPR) on a Biacore® T100 (GE Healthcare). C-terminally biotinylated IL-17 receptors were coupled to immobilized streptavidin on either an SA or CM4-sensor chip (GE Healthcare). An irrelevant, biotinylated protein was captured at equivalent immobilization densities to control flow cells. To measure the second receptor binding interaction, the cytokine was first captured to the immobilized receptor, followed by the second receptor injection. Low coupling densities (200-400 RU) and excess cytokine concentrations were used to optimize the number of cytokine homodimers bound to a single receptor. The surface was regenerated using 3 M MgCl2 between each cycle. For kinetic experiments, a flow rate of 50 μl/min was used. Data was analyzed using Biacore® T100 evaluation software Version 2.0 (GE Healthcare). Affinities are reported as the mean of at least two independent experiments±the standard error of the mean (s.e.m.).

### Example 21

### IL-17 Heterodimers Formed by Acid-Base Zippers

Several mutated IL-17 cytokine dimer proteins were designed as heterodimers of two different subunit sequences. One approach to preparing such heterodimers is by fusion of each respective subunit to one of two heterodimeric zipper sequences, e.g., one of a pair acidic-basic zippers. See, e.g., O'Shea et al., Curr Biol. (1993), 3(10):658-67. In this example, one subunit of IL-17A is expressed with a C-terminal tag that contained an acidic sequence and a hexahistidine tag. Another subunit of IL-17A is expressed with a C-terminal tag that contained a basic sequence and a hexahistidine tag. The sequence of these subunits is as follows:

IL-17A-Acid zipper:
(SEQ ID NO: 2)
GITIPRNPGCPNSEDKNFPRTVMVNLNIHNRNTNTNPKRSSDYYNRSTSP
WNLHRNEDPERYPSVIWEAKCRHLGCINADGNVDYHMNSVPIQQEILVLR
REPPHCPNSFRLEKILVSVGCTCVTPIVHHVASGGGGSRGGLEVLFQGPE
FGGSTTAPSAQLEKELQALEKENAQLEWELQALEKELAQHHHHHH
IL-17A-Base zipper:
(SEQ ID NO: 3)
GITIPRNPGCPNSEDKNFPRTVMVNLNIHNRNTNTNPKRSSDYYNRSTSP
WNLHRNEDPERYPSVIWEAKCRHLGCINADGNVDYHMNSVPIQQEILVLR
REPPHCPNSFRLEKILVSVGCTCVTPIVHHVASGGGGSRGGLEVLFQGPE
FGGSTTAPSAQLKKKLQALKKKNAQLKWKLQALKKKLAQHHHHHH

The constructs are co-transfected into 293 cells, for example, and protein and recovered.

### Example 22

### IL-17 Heterodimers Formed by Single Chain Fusion

Another approach to preparing heterodimers is by covalently linking the two subunits using a flexible peptide linker and expressing them as a single polypeptide chain. An example of a single chain IL-17A molecule is as follows:

(SEQ ID NO: 4)
GITIPRNPGCPNSEDKNFPRTVMVNLNIHNRNTNTNPKRSSDYYNRSTSP
WNLHRNEDPERYPSVIWEAKCRHLGCINADGNVDYHMNSVPIQQEILVLR
REPPHCPNSFRLEKILVSVGCTCVTPIVHHVASGGGGSGGGGSGGGGSGG
GGSGGGGSGITIPRNPGCPNSEDKNFPRTVMVNLNIHNRNTNTNPKRSSD
YYNRSTSPWNLHRNEDPERYPSVIWEAKCRHLGCINADGNVDYHMNSVPI
QQEILVLRREPPHCPNSFRLEKILVSVGCTCVTPIVHHVASHHHHHH

This protein is, for example, expressed in 293 cells. Supernatants from the cells were run on non-reducing gels and Western blot analysis using an anti-hexahistidine antibody is performed.

### Example 23

### Assay for IL-17 Activity

Control IL-17A and IL-17F proteins and mutant IL-17A and IL-17F proteins were evaluated in a cell-based functional assay according to the method of Fossiez et al., J. Exp. Med. 183(6):2593-603 (1996). Briefly, MRC-5 human embryonic fibroblast cells were subcultured in 96-well plates at a concentration of 1×10⁵ cells/well in DMEM with 10% FBS. Control proteins and proteins of interest in PBS, pH 7.4, were added to respective wells at a final concentration of 0.1-10,000 ng/mL. Cells were incubated an additional 48 hours. IL-6 concentration in the supernatants was then measured by ELISA using the Thermo Scientific Human IL-6 Screening Set (cat#ENESS0005). Using this assay, IL-17A and IL-17F control proteins were observed to have an EC50 within published ranges of 1-10 ng/mL for IL-17A and 50-100 ng/mL for IL-17F.

1-6. (canceled)

7. An IL-17 heterodimer comprising three subunits, wherein the first subunit is selected from the group consisting of a wild-type IL-17 monomer and an IL-17 monomer mutein, wherein the second subunit is an IL-17 monomer mutein, and wherein the third subunit is a linker.

8. The IL-17 heterodimer of claim 7, wherein the first and second subunits are derived from an IL-17 selected from the group consisting of IL-17A, IL-17B, IL-17C, IL-17D, IL-17E, and IL-17F.

9. The IL-17 heterodimer of claim 8, wherein the first subunit is derived from IL-17A and the second subunit is derived from IL-17F.

10. The IL-17 heterodimer of claim 7, wherein the first subunit is an IL-17 monomer mutein that comprises at least one amino acid substitution at a position selected from the group consisting of 89 and 95, wherein the numbering corresponds to IL-17F.

11. The IL-17 heterodimer of claim 7, wherein the second subunit comprises at least one amino acid substitution at a position selected from the group consisting of 41, 42, 47, 63, and 68, wherein the numbering corresponds to IL-17F.

12. The IL-17 heterodimer of claim 7, wherein the first subunit is an IL-17 monomer mutein that comprises at least one amino acid substitution at a position selected from the group consisting of 89 and 95, and wherein the second subunit comprises at least one amino acid substitution at a position selected from the group consisting of 41, 42, 47, 63, and 68, wherein the numbering corresponds to IL-17F.

13. The IL-17 heterodimer of claim 7, wherein the linker comprises a repeating sequence, wherein the repeating sequence encodes the polypeptide [(gly)B(ser)]X, wherein B is an integer from 1-5 and X is an integer from 1-20.

14. A nucleic acid encoding an IL-17 heterodimer comprising three subunits, wherein the first subunit is selected from the group consisting of a wild-type IL-17 monomer and an IL-17 monomer mutein, wherein the second subunit is an IL-17 monomer mutein, and wherein the third subunit is a linker.

15. The nucleic acid of claim 14, wherein the first and second subunits are derived from an IL-17 selected from the group consisting of IL-17A, IL-17B, IL-17C, IL-17D, IL-17E, and IL-17F.

16. The nucleic acid of claim 14, wherein the first subunit is derived from IL-17A and the second subunit is derived from IL-17F.

17. The nucleic acid of claim 14, wherein the first subunit is an IL-17 monomer mutein that comprises at least one amino acid substitution at a position selected from the group consisting of 89 and 95, wherein the numbering corresponds to IL-17F.

18. The nucleic acid of claim 14, wherein the second subunit comprises at least one amino acid substitution at a position selected from the group consisting of 41, 42, 47, 63, and 68, wherein the numbering corresponds to IL-17F.

19. The nucleic acid of claim 14, wherein the first subunit is an IL-17 monomer mutein that comprises at least one amino acid substitution at a position selected from the group consisting of 89 and 95, and wherein the second subunit comprises at least one amino acid substitution at a position selected from the group consisting of 41, 42, 47, 63, and 68, wherein the numbering corresponds to IL-17F.

20. The nucleic acid of claim 14, wherein the linker comprises a repeating sequence, wherein the repeating sequence encodes the polypeptide [(gly)B(ser)]X, wherein B is an integer from 1-5 and X is an integer from 1-20.

21. An isolated host cell comprising the nucleic acid of claim 14

22. A method for preparing an IL-17 heterodimer protein, the method comprising obtaining a host cell that comprises the nucleic acid of claim 14, growing the host cell in a host cell culture, providing host cell culture conditions wherein the nucleic acid is expressed, and recovering the IL-17 heterodimer protein from the host cell or the host cell culture.

23. A pharmaceutical composition comprising the IL-17 heterodimer protein of claim 7.

24. A method of treating an IL-17 mediated disorder in a subject, wherein the method comprises administering to the subject an effective amount of the pharmaceutical composition of claim 23.

25. The method of claim 24, wherein the IL-17 mediated disorder is selected from the group consisting of airway inflammation, rheumatoid arthritis (“RA”), osteoarthritis, bone erosion, intraperitoneal abscesses and adhesions, inflammatory bowel disorder (“IBD”), allograft rejection, psoriasis, cancer, angiogenesis, atherosclerosis and multiple sclerosis (“MS”).
